(19) United States
(12) Patent Application Publication
(10) Pub. No.: US 2022/0023301 A1
(43) Pub. Date: Jan. 27, 2022

(54) METHODS OF TREATING AUTOIMMUNE DISEASES WITH SMALL MOLECULE NF-kB INHIBITORS

(71) Applicant: ImmuneTarget, Inc., Medina, WA (US)

(72) Inventors: Samedy Ouk, Medina, WA (US); Hsiou-Chi Liou, New York, NY (US)

(73) Assignee: ImmuneTarget, Inc., Medina, WA (US)

(21) Appl. No.: 17/384,661

(22) Filed: Jul. 23, 2021

Related U.S. Application Data
(60) Provisional application No. 63/057,167, filed on Jul. 27, 2020.

(51) Int. Cl. A61K 31/519; A61P 37/06

(52) U.S. Cl. CPC A61K 31/519; A61P 37/06

(57) ABSTRACT

The present invention provides, inter alia, compounds capable of inhibiting NF-κB. Pharmaceutical compositions containing and methods of using the compounds are also provided herein. Also provided are compositions, methods and kits for treating autoimmune diseases, including but not limited to rheumatoid arthritis, multiple sclerosis, systemic lupus erythematosus, all comprising and/or utilizing the NF-κB inhibitors described herein.

### CROSS-REFERENCE TO RELATED APPLICATIONS

This application claims priority to U.S. Provisional Application Ser. No. 63/057,167, filed Jul. 27, 2020, the disclosure of which is hereby incorporated by reference.

### FIELD OF INVENTION

The present invention provides, inter alia, compounds and pharmaceutical compositions capable of inhibiting NF-κB, as well methods of using said compounds to treat autoimmune diseases and compositions.

### BACKGROUND OF THE INVENTION

### NF-κB/Rel

NF-κB/Rel (nuclear factor kappa B) is a family of transcription factors that includes p50/p105 (NF-κB1), p52/p100 (NF-κB2), p65 (RelA), c-Rel, and RelB. These molecules can homo- or heterodimerize; and are generally sequestered in the cytoplasm by their inhibitors, IκBs. Upon activation, IκBs are degraded by the 26s proteasome and NF-κB dimers migrate into the nucleus to perform transcriptional activity.

NF-κB (p50/p65) and c-Rel are regulated by the canonical IKK α/β/γ kinase complex pathway, whereas RelB and p52 (NF-κB2) are regulated by an alternative pathway via the IKKα/NIK complex. Despite this similarity, each NF-κB family member is distinct with regard to tissue expression pattern, response to receptor signals, and target gene specificity. These differences are evident from the non-redundant phenotypes exhibited by individual NF-κB/Rel knockout mice. Therefore, therapeutics targeted to different NF-κB/Rel members are likely to have different biological effects and toxicity profiles.

Many receptors and stimuli can activate NF-κB/Rel, including TCR/BCR, TNF receptor superfamily (e.g. CD40, TNFR1, TNFR2, BAFF, APRIL, RANK), IL-1/TLR receptors, and Nod-like receptors, as well as activating oncogenes (e.g. Src, Ras, LMP-1, Tax, v-FLIP), reactive oxygen radicals, radiation, and chemotherapeutic agents. In response to these stimuli, NF-κB/Rel regulates the expression of cytokines, chemokines, and molecules that play a role in adhesion, the cell cycle, apoptosis, immune modulation, and angiogenesis. As such, NF-κB/Rel transcription factors are important therapeutic targets for many human disorders, including inflammation, autoimmune diseases, and cancer, and small molecule inhibitors of NF-κB/Rel may be useful as therapeutics for these disorders.

Many human diseases including inflammation, autoimmune disease, and cancer are attributed to aberrant activation of transcription factors, which leads to dysregulated target gene expression and evidence of new biological activities as well as survival or proliferative advantages of residential cell types. In the transcription factor field, NF-kB has attracted central attention as being a transcription factor that is involved in a myriad of biological functions and pathological conditions including the regulation of innate and adaptive immune response to infection, inflammation, cell survival, and tumorigenesis.

Rel/NF-kB as drug target for rheumatoid arthritis (RA)

RA is characterized by the production of inflammatory cytokines/mediators in synovial tissues, many of which are regulated by Rel/NF-kB transcription factors. REL or NFKB1 knockout mice are protected from Collagen Induced Arthritis, a clinically relevant model commonly used in RA drug development [I. Campbell, et al. J Clin Invest. 2000, 105(12), 1799].

Drugs used to manage RA target both upstream and downstream of NF-kB pathways, from the oldest glucocorticoid, to the most popular anti-TNF adalimumab, to the newest JAK inhibitor upadacitinib. Although many disease-modifying anti-rheumatic drugs (DMARDs) have been approved, RA remains incurable and often refractory to treatment, hence the need of a new class of immunomodulator drug.

Historically, for pain relief and reduce inflammation caused by RA, patients turned to plant extracts such as salicin, the historical precursor of aspirin, and Tanacetum parthenium, aka Feverfew, an ancient anti-inflammatory remedy which contains parthenolide as an active ingredient. Modern science has proved that parthenolide binds to, and inhibits Rel/NF-kB [S. Ouk et al. Future Med. Chem. 2009, 1(9), 1683]. Similarly, (−)-dehydroxymethyl epoxyquinomycin (DHMEQ), a covalently-bond NF-kB inhibitor, was proven to be an effective anti-inflammatory and anti-cancer compound in many animal models. However, DHMEQ's poor pharmacokinetics (PK) limits its clinical use [K. Umezawa, et al. Biomed. Pharmacother. 2011, 65, 252]. Its instability is due to its reactive epoxy activity, the very function required for its ability to bind to Rel/NF-kB.

Rel/NF-kB as Drug Target for Multiple Sclerosis (MS)

Multiple sclerosis (MS) is an inflammatory central nervous system (CNS) in which immune system causes demyelination of neuron's axon. The damage of myelin sheet that protects axon of nerve cells leads to inefficient and loss of nerve impulse. Hence, patient may experience different symptoms such as pain, tremor, impaired movement coordination, poor vision and fatigue.

A very large genome-wide association studies and one meta-analysis successfully identified 57 non-HLA susceptibility loci. Further search for subnetworks (modules) of genes and their encoding proteins indicated that these modules were more likely to contain genes with bona fide susceptibility variants. In particular, the study identified several high-confidence candidates (including BCL10, CD48, REL, TRAF3, and TEC) [International Multiple Sclerosis Genetics Consortium, Am. J. Hum. Genet. 2013, 92, 854-865].

Conditional knockout-mice for IκBα in myeloid cells (lysMCreIκBα(fl/fl)) have been generated and are characterized by a constitutive activation of NF-κB proteins allowing the study of this transcription factor in myelin-oligodendrocyte-glycoprotein induced experimental autoimmune encephalomyelitis (MOG-EAE), a well-established experimental model for autoimmune demyelination of the CNS. Compared to controls, lysMCreIκBα(fl/fl) mice developed a more severe clinical course of EAE. In addition, lysMCreIκBα(fl/fl) mice displayed an increased expression of the NF-κB dependent factor inducible nitric oxide synthase in inflamed lesions. These changes in the CNS are associated with increased numbers of CD11b positive splenocytes and a higher expression of Ly6c on monocytes in the periphery. In accordance with these changes in the myeloid cell compartment, there was an increased production of the monocyte cytokines interleukin (IL)-12 p70, IL-6 and IL-1beta in splenocytes. By contrast, production of the T-cell associated cytokines interferon gamma (IFN-gamma) and IL-17 was not affected. In summary, myeloid cell derived NF-κB plays a crucial role in autoimmune inflammation of the CNS and drives a pathogenic role of monocytes and macrophages independent of T-cells [G. Ellrichman et al. J. Neuroinflammation 2012, 9:15].

In light of genetic evidence from a large cohort of MS patients and the development of EAE in lysMCreIκBα(fl/fl) mice, we believe that inhibition of Rel/NF-kB has potential to manage MS symptom, prevent further damage to myelin, and repair myelin sheet. Indeed, natural product Triptolide, an NF-κB inhibitor, at 0.125 mg/kg administered intraperitoneally daily, improves motor function and motor coordination compared to control group in cuprizone induced neuroinflammation mouse model. It appears that triptolide at 0.125 mg/kg dose was able to raise the number of OLGs precursor cells (NG-2⁺/O4⁺), reduce Bax/Bcl-2 ratio, and improve behavioral deficits [N. Sanadgol et al. Toxico. Appl. Pharmacol. 2018, 842, 86].

Rel/NF-kB as Drug Target for Systemic Lupus Erythematosus (SLE)

Systemic lupus erythematosus (SLE) is a chronic autoimmune disease characterized by multi-organ inflammation, resulting from loss of tolerance to self-antigens and production of anti-nuclear antibodies. These antibody-nuclear antigen complexes drive inflammation in multiple organs, including the kidney, resulting in tissue damage. It is thought that nucleic acid-immune complexes activate the innate immune response through Toll-like receptors (TLR) 7 and 9 in plasmacytoid dendritic cells (pDC) and other cell types, driving production of type I interferon. The resulting interferon signature metric not only correlates with SLE disease severity in human patients, but also promotes disease, as blockade of type I interferon signaling through anifrolumab has shown promising efficacy in a phase II clinical trial.

In addition to TLR and type I interferon (IFN), several tumor necrosis factor (TNF) receptor superfamily (TNFRSF) members are implicated in SLE pathophysiology. B cell-activating factor (BAFF) and CD40 are required for B cell survival and differentiation to auto-antibody-producing plasma cells. Blockade of CD40 ligand (CD40L) showed promising effects in early lupus clinical trials, even though development was halted due to thrombotic side effects. On the other hand, BAFF blockade through belimumab is partially efficacious and is the only new therapy for lupus approved in more than 50 years [H. D. Brightbill, et. al. Nature Comm. 2018; 19:179] [R. K. Mishra Nephrol Open J. 2016; 2(1): 9-13].

Notably, nuclear NF-kB is highly activated by the aforementioned signaling pathways, including TLR7, TLR9, CD40, and BAFF, during SLE pathogenesis. To investigate the role of NF-κB family members NF-1κB1, NF-κB2 and c-REL in SLE. L. A. O'Reilly et al. generated Fas^{lpr/lpr} mutant mice and showed that loss of each of these transcription factors resulted in amelioration of many classical features of autoimmune disease, including hypergammaglobulinaemia, anti-nuclear autoantibodies and autoantibodies against tissue-specific antigens. Remarkably, only c-REL deficiency substantially reduced immune complex-mediated glomerulonephritis and extended the lifespan of Fas^{lpr/lpr} mice. The findings demonstrate that different NF-1B family members exert distinct roles in the development of the diverse autoimmune and lymphoproliferative pathologies that arise in Fas^{lpr/lpr} mice, and suggest that pharmacological targeting of c-REL should be considered as a strategy for therapeutic intervention in autoimmune diseases [J. L. Low et al. Immunology and Cell Biology 2016, 94, 66-78]

In addition to the aforementioned diseases (rheumatoid arthritis, multiple sclerosis, Systemic lupus erythematosus), other autoimmune disorders associated with Rel/NF-kB activation include Type I diabetes, Ankylosing spondylitis, Spondyloarthropathies, Crohn's disease (inflammatory bowel disease), Grave's disease, Hashimoto's thyroiditis, Myasthenia gravis, Psoriasis, Lymphoproliferative disease (ALPS), Sjogren's syndrome, Autoimmune neuropathies, Gullian-Barre syndrome, Autoimmune uveitis, Autoimmune hemolytic anemia, Pernicious anemia, Aplastic anemia, Pure red cell anemia, Autoimmune thrombocytopenia, Temporal arteritis, Anti-phospholipid syndrome, Vasculitides, Wegener's granulomatosis, Behcet's disease, Dermatitis herpetiformis, Pemphigus vulgaris, Vitiligo, Primary biliary cirrhosis, Autoimmune hepatitis, Autoimmune oophoritis and orchitis, Autoimmune disease of the adrenal gland, Scleroderma, Polymyositis, Dermatomyositis, Autoimmune menagitis, Autoimmune dermatitis, Alopecia areata, Autoimmune uveitis, Allergic encephalomyelitis, Interstitial lung fibrosis, Seronegative arthropathies, Sarcoidosis, Orchitis/vasectomy reversal procedure, Raynoud's disease, Type B insulin-resistant diabetes, Antibody-mediated cytotoxicity, Type III hypersensitivity reactions, POEMS syndrome, Polyneuropathy, Organomegaly, Endocrinopathy, Monoclonal gammopathy, Skin changes syndrome, Pemphigus, Mixed connective tissue diseases, Idiopathic Addison's disease, Post-MI cardiotomy syndrome, Wilson's disease, Hemachromatosis, Alpha-1-antitrypsin deficiency, Osteoporosis, Hypothalamic-pituitary-adrenal axis evaluation, Familial hematophagocytic lymphohistiocytosis, Pre-eclampsia, OKT3 therapy, Anti-CD3 therapy, Cytokine therapy, Chemotherapy, Radiation therapy and Immune tolerance therapy via co-administration of self-antigens or self-tissues.

Rel activation has also been implicated in a wide variety of diseases and pathological conditions, including AIDS, diabetes mellitus, cardiovascular diseases, atherosclerosis, septic shock syndrome, viral replication, osteoporosis, bone loss, organ transplant rejection, graft-versus-host diseases (GVHD), neurodegenerative disorders, ataxia telangiectasia, metabolic disorders, type 1 and type 2 diabetes, as well as aging. Specifically, the c-Rel knockout mice studies have clearly demonstrated the involvement of c-Rel activation in stress-induced atherosclerosis (A. Bierhaus et. al. 2010. JCI) and transplant rejection (Finn P W et. al. 2001, JI; Finn, P W et. al. 2002, J Leukoc. Biol; Yang H. et. al. Transplantation, 2002).

### SUMMARY OF THE INVENTION

The present invention relates to compounds capable of inhibiting NF-kB/Rel.

One embodiment of the present invention is a compound. The compound has a structure of formula (I)

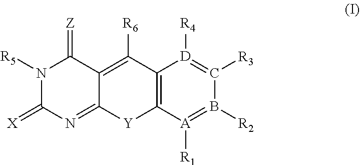

wherein:
A, B, C, and D are independently selected from the group consisting of carbon and nitrogen;
X, Y, and Z are independently selected from the group consisting of oxygen, sulfur, and NR^{a};
R₁, R₂, R₃, and R₄ are independently selected from the group consisting of no atom, hydrogen, halogen, C₁₋₉ alkyl, C₂₋₉ alkenyl, C₂₋₉ alkynyl, aryl, heterocyclic, —OH, —OR^{a}, —OR^{a}OR^{b}, —OR^{a}OR^{b}OR^{c}, —OR^{a}(C═O)R^{b}, —O(C═O)R^{a}, —O(C═O)OR^{a}, —O(C═O)NR^{a}R^{b}, cyano, nitro, —CF₃, —CHF₂, —CH₂F, —CHO, —COOH, —COR^{a}, —COOR^{a}, —CONR^{a}R^{b}, —CONHCONR^{a}R^{b}, —NR^{a}R^{b}, —NHCOR^{a}, —NR^{b}COR^{a}, —CSOH, —CSR^{a}, —CSOR^{a}, —CSNR^{a}R^{b}, —CSNHCSNR^{a}R^{b}, —SH, —SR^{a}, —S(C═O)R^{a}, —S(C═O)OR^{a}, —S(C═O)NR^{a}R^{b};
R₅ is selected from the group consisting of hydrogen, C₁₋₉ alkyl, C₂₋₉ alkenyl, C₂₋₉ alkynyl, aryl, heterocyclic, —R^{a}CO, —R^{a}NHCO, and —R^{a}OCO; and
R₆, R^{a}, R^{b}, and R^{c} are independently selected from the group consisting of hydrogen, hydroxyl, amine, C₁₋₉ alkyl, C₂₋₉ alkenyl, C₂₋₉ alkynyl, aryl, and heterocyclic,
or is a crystalline form, hydrate, or pharmaceutically acceptable salt thereof.

Another embodiment of the present invention is a compound. The compound is selected from the group consisting of the compounds in Table 1, and crystalline forms, hydrates, or pharmaceutically acceptable salts thereof.

TABLE 1

| Designation | Structure |
|---|---|
| IT-806 | 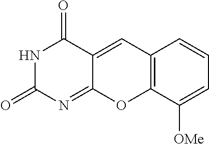 [SMILES: COc1cccc2cc3c(=O)[nH]c(=O)nc-3oc12] |
| IT-807 | 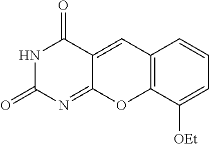 [SMILES: CCOc1cccc2cc3c(=O)[nH]c(=O)nc-3oc12] |
| IT-809 | 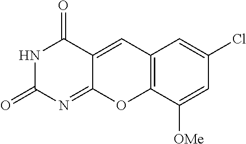 [SMILES: COc1cc(Cl)cc2cc3c(=O)[nH]c(=O)nc-3oc12] |
| IT-810 | 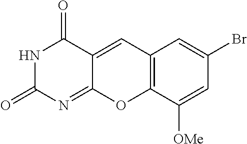 [SMILES: COc1cc(Br)cc2cc3c(=O)[nH]c(=O)nc-3oc12] |
| IT-814 | 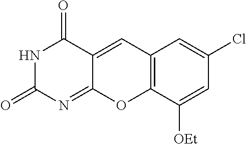 [SMILES: CCOc1cc(Cl)cc2cc3c(=O)[nH]c(=O)nc-3oc12] |
| IT-817 | 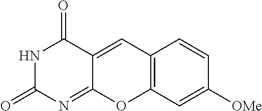 [SMILES: COc1ccc2cc3c(=O)[nH]c(=O)nc-3oc2c1] |
| IT-854 | 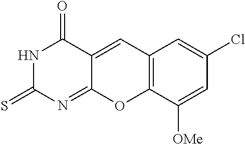 [SMILES: COc1cc(Cl)cc2cc3c(=O)[nH]c(=S)nc-3oc12] |
| IT-861 | 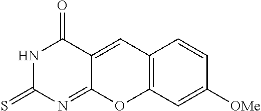 [SMILES: COc1ccc2cc3c(=O)[nH]c(=S)nc-3oc2c1] |
| IT-804 | 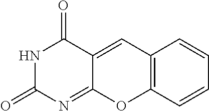 [SMILES: O=c1nc2oc3ccccc3cc-2c(=O)[nH]1] |
| IT-818 | 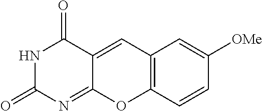 [SMILES: COc1ccc2oc3nc(=O)[nH]c(=O)c-3cc2c1] |
| IT-819 | 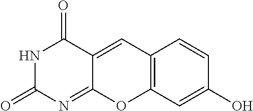 [SMILES: O=c1nc2oc3cc(O)ccc3cc-2c(=O)[nH]1] |
| IT-820 | 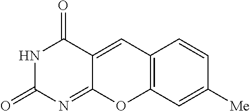 [SMILES: Cc1ccc2cc3c(=O)[nH]c(=O)nc-3oc2c1] |
| IT-821 | 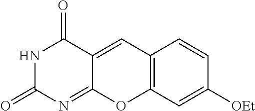 [SMILES: CCOc1ccc2cc3c(=O)[nH]c(=O)nc-3oc2c1] |
| IT-822 | 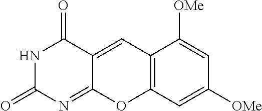 [SMILES: COc1cc(OC)c2cc3c(=O)[nH]c(=O)nc-3oc2c1] |
| IT-862 | 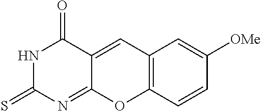 [SMILES: COc1ccc2oc3nc(=S)[nH]c(=O)c-3cc2c1] |
| IT-863 | 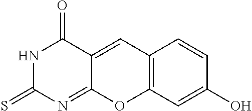 [SMILES: O=c1[nH]c(=S)nc2oc3cc(O)ccc3cc1-2] |
| IT-864 | 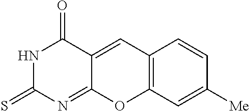 [SMILES: Cc1ccc2cc3c(=O)[nH]c(=S)nc-3oc2c1] |
| IT-865 | 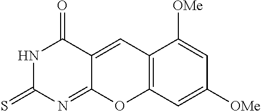 [SMILES: COc1cc(OC)c2cc3c(=O)[nH]c(=S)nc-3oc2c1] |
| IT-823 | 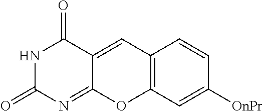 |
| IT-826 | 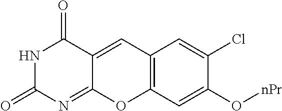 |
| IT-825 | 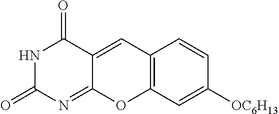 |
| IT-827 | 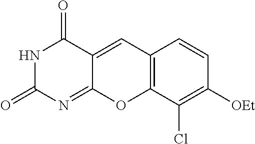 [SMILES: CCOc1ccc2cc3c(=O)[nH]c(=O)nc-3oc2c1Cl] |
| IT-824 | 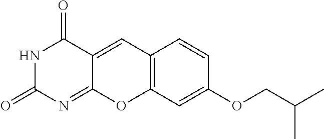 [SMILES: CC(C)COc1ccc2cc3c(=O)[nH]c(=O)nc-3oc2c1] |
| IT-828 | 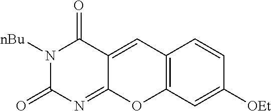 |
| IT-829 | 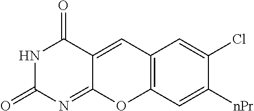 |
| IT-830 | 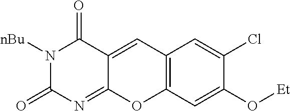 |
| IT-831 | 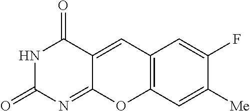 [SMILES: Cc1cc2oc3nc(=O)[nH]c(=O)c-3cc2cc1F] |
| IT-832 | 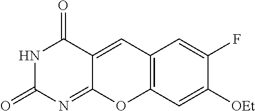 [SMILES: CCOc1cc2oc3nc(=O)[nH]c(=O)c-3cc2cc1F] |
| IT-833 | 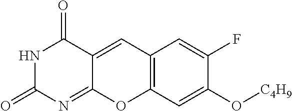 |
| IT-834 | 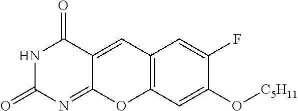 |
| IT-835 | 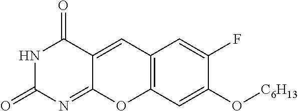 |
| IT-836 | 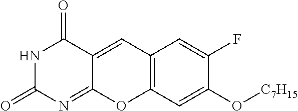 |
| IT-837 | 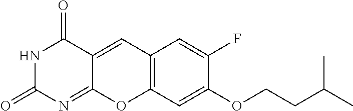 [SMILES: CC(C)CCOc1cc2oc3nc(=O)[nH]c(=O)c-3cc2cc1F] |
| IT-838 | 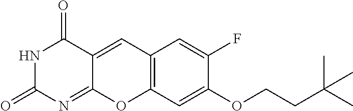 [SMILES: CC(C)(C)CCOc1cc2oc3nc(=O)[nH]c(=O)c-3cc2cc1F] |
| IT-839 | 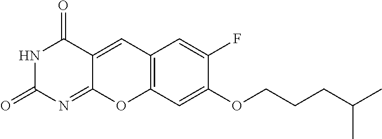 [SMILES: CC(C)CCCOc1cc2oc3nc(=O)[nH]c(=O)c-3cc2cc1F] |
| IT-840 | 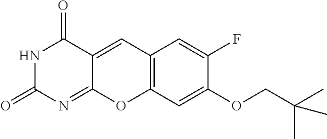 [SMILES: CC(C)(C)COc1cc2oc3nc(=O)[nH]c(=O)c-3cc2cc1F] |
| IT-841 | 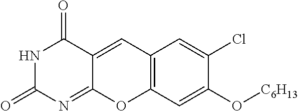 |
| IT-842 | 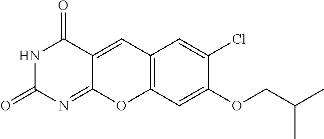 [SMILES: CC(C)COc1cc2oc3nc(=O)[nH]c(=O)c-3cc2cc1Cl] |
| IT-843 | 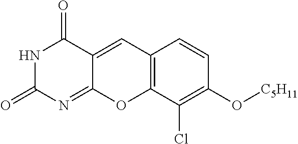 |
| IT-844 | 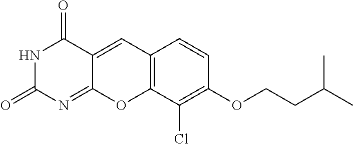 [SMILES: CC(C)CCOc1ccc2cc3c(=O)[nH]c(=O)nc-3oc2c1Cl] |
| IT-845 | 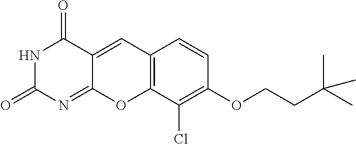 [SMILES: CC(C)(C)CCOc1ccc2cc3c(=O)[nH]c(=O)nc-3oc2c1Cl] |
| IT-846 | 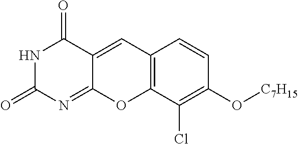 |
| IT-847 | 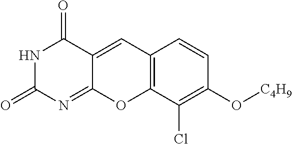 |
| IT-848 | 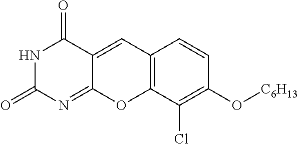 |
| IT-849 | 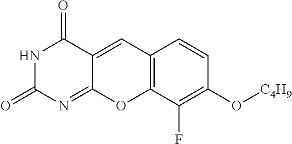 |
| IT-850 | 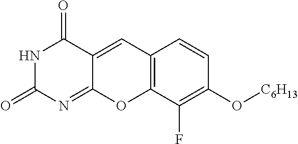 |
| IT-851 | 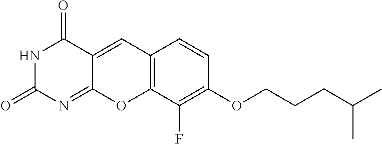 [SMILES: CC(C)CCCOc1ccc2cc3c(=O)[nH]c(=O)nc-3oc2c1F] |
| IT-852 | 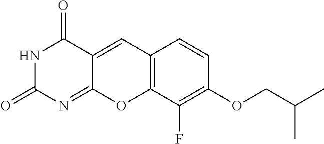 [SMILES: CC(C)COc1ccc2cc3c(=O)[nH]c(=O)nc-3oc2c1F] |
| IT-872 | 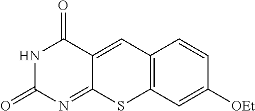 [SMILES: CCOc1ccc2cc3c(=O)[nH]c(=O)nc-3sc2c1] |
| IT-882 | 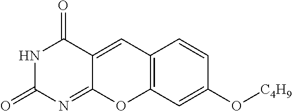 |
| IT-883 | 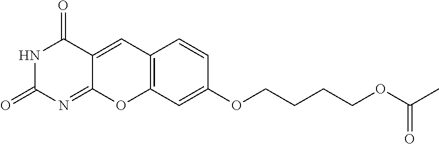 [SMILES: CC(=O)OCCCCOc1ccc2cc3c(=O)[nH]c(=O)nc-3oc2c1] |
| IT-853 | 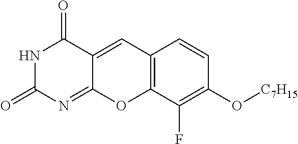 |
| IT-855 | 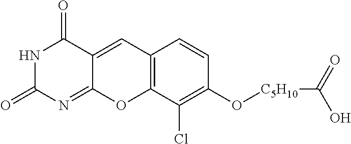 |
| IT-856 | 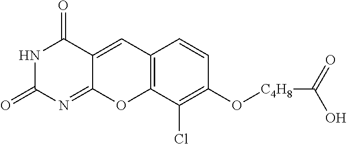 |
| IT-857 | 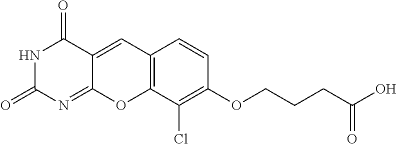 [SMILES: O=C(O)CCCOc1ccc2cc3c(=O)[nH]c(=O)nc-3oc2c1Cl] |
| IT-858 | 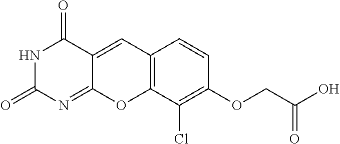 [SMILES: O=C(O)COc1ccc2cc3c(=O)[nH]c(=O)nc-3oc2c1Cl] |
| IT-859 | 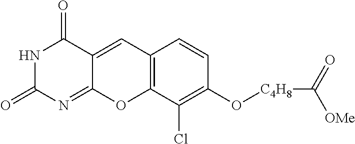 |
| IT-866 | 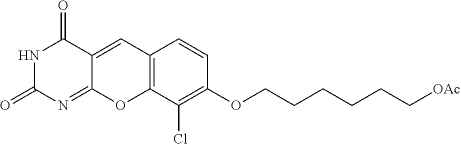 [SMILES: CC(=O)OCCCCCCOc1ccc2cc3c(=O)[nH]c(=O)nc-3oc2c1Cl] |
| IT-867 | 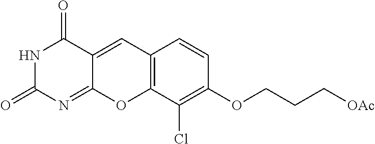 [SMILES: CC(=O)OCCCOc1ccc2cc3c(=O)[nH]c(=O)nc-3oc2c1Cl] |
| IT-868 | 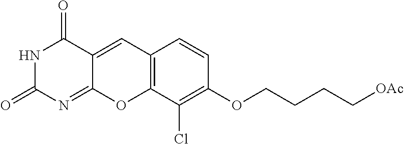 [SMILES: CC(=O)OCCCCOc1ccc2cc3c(=O)[nH]c(=O)nc-3oc2c1Cl] |
| IT-875 | 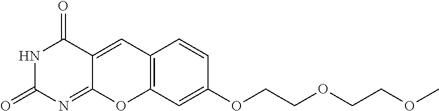 [SMILES: COCCOCCOc1ccc2cc3c(=O)[nH]c(=O)nc-3oc2c1] |
| IT-876 | 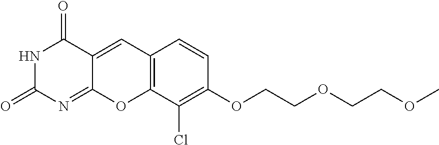 [SMILES: COCCOCCOc1ccc2cc3c(=O)[nH]c(=O)nc-3oc2c1Cl] |
| IT-884 | 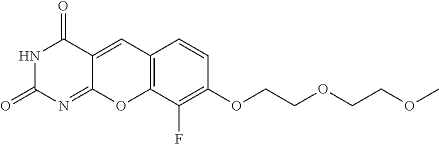 [SMILES: COCCOCCOc1ccc2cc3c(=O)[nH]c(=O)nc-3oc2c1F] |
| IT-885 | 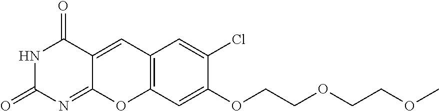 [SMILES: COCCOCCOc1cc2oc3nc(=O)[nH]c(=O)c-3cc2cc1Cl] |
| IT-886 | 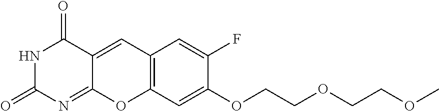 [SMILES: COCCOCCOc1cc2oc3nc(=O)[nH]c(=O)c-3cc2cc1F] |
| IT-877 | 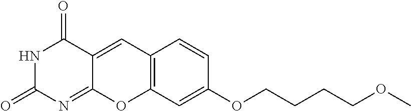 [SMILES: COCCCCOc1ccc2cc3c(=O)[nH]c(=O)nc-3oc2c1] |
| IT-878 | 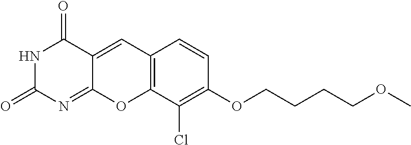 [SMILES: COCCCCOc1ccc2cc3c(=O)[nH]c(=O)nc-3oc2c1Cl] |
| IT-887 | 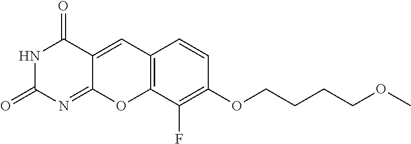 [SMILES: COCCCCOc1ccc2cc3c(=O)[nH]c(=O)nc-3oc2c1F] |
| IT-888 | 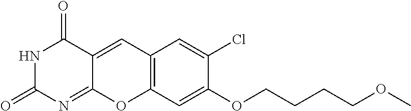 [SMILES: COCCCCOc1cc2oc3nc(=O)[nH]c(=O)c-3cc2cc1Cl] |
| IT-889 | 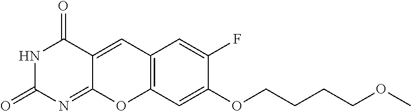 [SMILES: COCCCCOc1cc2oc3nc(=O)[nH]c(=O)c-3cc2cc1F] |
| IT-890 | 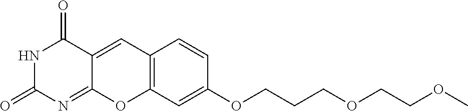 [SMILES: COCCOCCCOc1ccc2cc3c(=O)[nH]c(=O)nc-3oc2c1] |
| IT-879 | 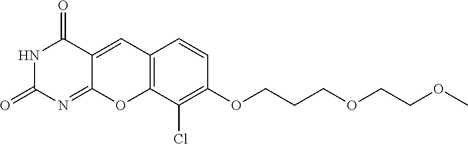 [SMILES: COCCOCCCOc1ccc2cc3c(=O)[nH]c(=O)nc-3oc2c1Cl] |
| IT-891 | 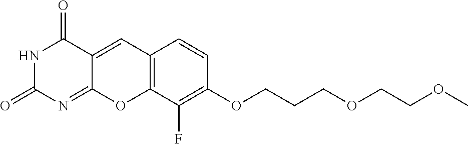 [SMILES: COCCOCCCOc1ccc2cc3c(=O)[nH]c(=O)nc-3oc2c1F] |
| IT-892 | 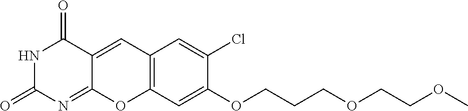 [SMILES: COCCOCCCOc1cc2oc3nc(=O)[nH]c(=O)c-3cc2cc1Cl] |
| IT-880 | 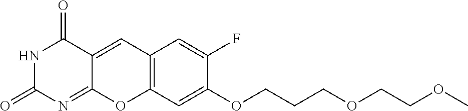 [SMILES: COCCOCCCOc1cc2oc3nc(=O)[nH]c(=O)c-3cc2cc1F] |
| IT-893 | 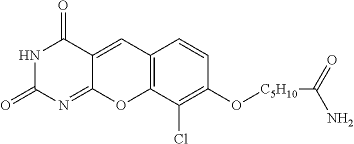 |
| IT-894 | 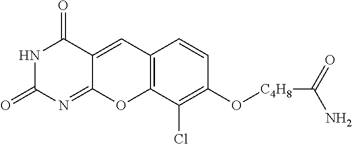 |
| IT-895 | 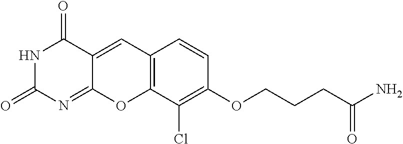 [SMILES: NC(=O)CCCOc1ccc2cc3c(=O)[nH]c(=O)nc-3oc2c1Cl] |
| IT 896 | 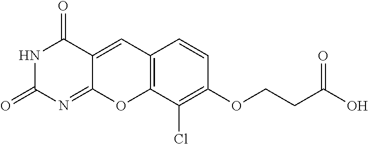 [SMILES: O=C(O)CCOc1ccc2cc3c(=O)[nH]c(=O)nc-3oc2c1Cl] |
| IT-897 | 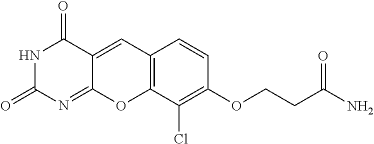 [SMILES: NC(=O)CCOc1ccc2cc3c(=O)[nH]c(=O)nc-3oc2c1Cl] |
| IT-898 | 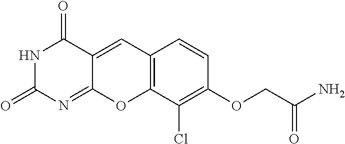 [SMILES: NC(=O)COc1ccc2cc3c(=O)[nH]c(=O)nc-3oc2c1Cl] |
| IT-899 | 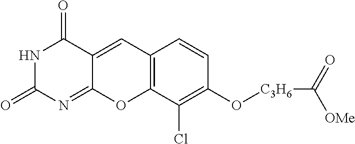 |
| IT-701 | 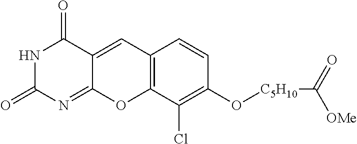 |
| IT-702 | 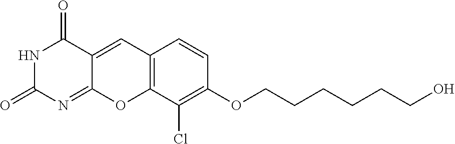 [SMILES: O=c1nc2oc3c(Cl)c(OCCCCCCO)ccc3cc-2c(=O)[nH]1] |
| IT-703 | 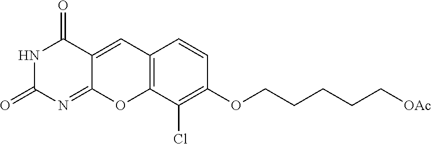 [SMILES: CC(=O)OCCCCCOc1ccc2cc3c(=O)[nH]c(=O)nc-3oc2c1Cl] |
| IT-704 | 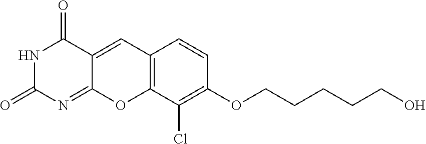 [SMILES: O=c1nc2oc3c(Cl)c(OCCCCCO)ccc3cc-2c(=O)[nH]1] |
| IT-705 | 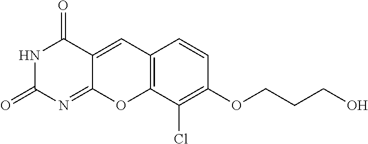 [SMILES: O=c1nc2oc3c(Cl)c(OCCCO)ccc3cc-2c(=O)[nH]1] |
| IT-706 | 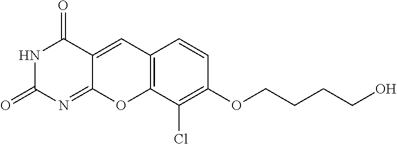 [SMILES: O=c1nc2oc3c(Cl)c(OCCCCO)ccc3cc-2c(=O)[nH]1] |
| IT-707 | 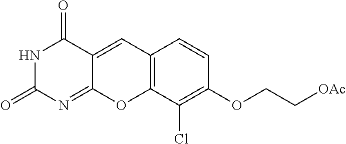 [SMILES: CC(=O)OCCOc1ccc2cc3c(=O)[nH]c(=O)nc-3oc2c1Cl] |
| IT-708 | 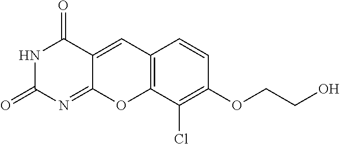 [SMILES: O=c1nc2oc3c(Cl)c(OCCO)ccc3cc-2c(=O)[nH]1] |

A further embodiment of the present invention is a compound. The compound is selected from the group consisting of:

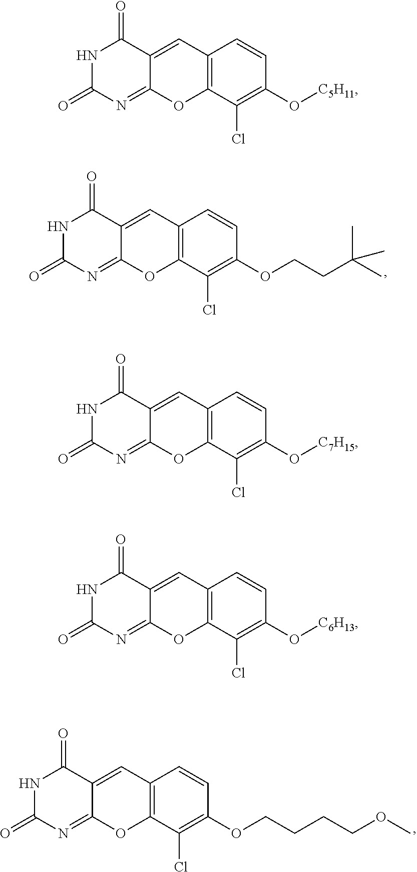

and crystalline forms, hydrates, and pharmaceutically acceptable salts thereof.

An additional embodiment of the present invention is a compound capable of inhibiting NF-κB. The compound has the structure of formula (I):

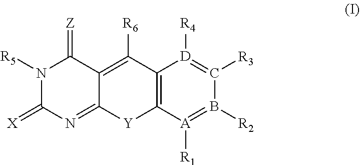

wherein:
A, B, C, and D are independently selected from the group consisting of carbon and nitrogen;
X, Y, and Z are independently selected from the group consisting of oxygen, sulfur, and NR^{a};
R₁, R₂, R₃, and R₄ are independently selected from the group consisting of no atom, hydrogen, halogen, C₁₋₉ alkyl, C₂₋₉ alkenyl, C₂₋₉ alkynyl, aryl, heterocyclic, —OH, —OR^{a}, —OR^{a}OR^{b}, —OR^{a}OR^{b}OR^{c}, —O(C═O)R^{a}, —O(C═O)OR^{a}, —O(C═O)NR^{a}R^{b}, cyano, nitro, CF₃, CHF₂, CH₂F, —CHO, —COOH, —COR^{a}, —COOR^{a}, —CONR^{a}R^{b}, —CONHCONR^{a}R^{b}, —NR^{a}R^{b}, —NHCOR^{a}, —NR^{b}COR^{a}, —CSOH, —CSR^{a}, —CSOR^{a}, —CSNR^{a}R^{b}, —CSNHCSNR^{a}R^{b}, —SH, —SR^{a}, —S(C═O)R^{a}, —S(C═O)OR^{a}, —S(C═O)NR^{a}R^{b};
R₅ is selected from the group consisting of hydrogen, C₁₋₉ alkyl, C₂₋₉ alkenyl, C₂₋₉ alkynyl, aryl, heterocyclic, —R^{a}CO, —R^{a}NHCO, and —R^{a}OCO; and
R₆, R^{a}, R^{b}, and R^{c} are independently selected from the group consisting of hydrogen, hydroxyl, amine, C₁₋₉ alkyl, C₂₋₉ alkenyl, C₂₋₉ alkynyl, aryl, and heterocyclic,
   or is a crystalline form, hydrate, or pharmaceutically acceptable salt thereof.

A further embodiment of the present invention is a compound capable of inhibiting NF-κB. The compound is selected from the group consisting of the compounds in Table 1, and crystalline forms, hydrates, and pharmaceutically acceptable salts thereof.

An additional embodiment of the present invention is a compound capable of inhibiting NF-κB. The compound is selected from the group consisting of:

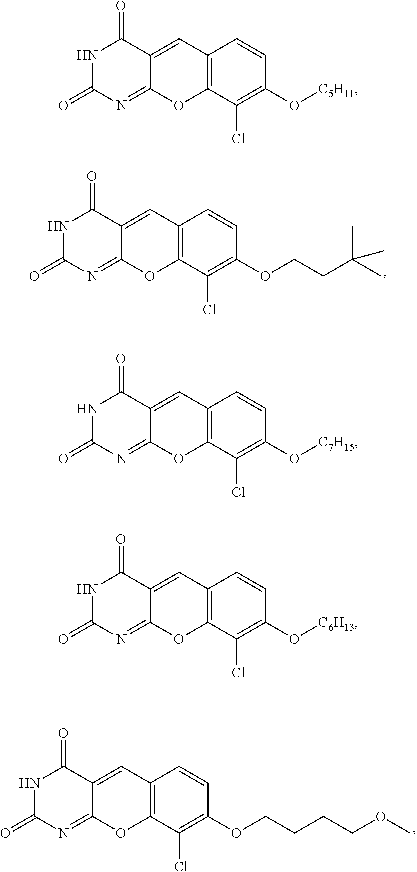

and crystalline forms, hydrates, and pharmaceutically acceptable salts thereof.

Another embodiment of the present invention is a pharmaceutical composition. The pharmaceutical composition comprises a pharmaceutically acceptable carrier and any of the compounds disclosed herein.

A further embodiment of the present invention is a method of inhibiting NF-κB in a cell. The method comprises contacting the cell with any of the compounds disclosed herein. In another embodiment, the method comprises contacting the cell with one of or a combination of any of the compounds disclosed herein.

An additional embodiment of the present invention is a method for treating autoimmune diseases in a subject. The method comprises administering to the subject an effective amount of a compound having the structure of formula (I):

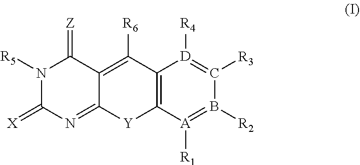

wherein:
A, B, C, and D are independently selected from the group consisting of carbon and nitrogen;
X, Y, and Z are independently selected from the group consisting of oxygen, sulfur, and NR^{a};
R₁, R₂, R₃, and R₄ are independently selected from the group consisting of no atom, hydrogen, halogen, C₁₋₉ alkyl, C₂₋₉ alkenyl, C₂₋₉ alkynyl, aryl, heterocyclic, —OH, —OR^{a}, —OR^{a}OR^{b}, —OR^{a}OR^{b}OR^{c}, —OR^{a}(C═O)R^{b}—O(C═O)R^{a}, —O(C═O)OR^{a}, —O(C═O)NR^{a}R^{b}, cyano, nitro, —CF₃, —CHF₂, —CH₂F, —CHO, —COOH, —COR^{a}, —COOR^{a}, —CONR^{a}R^{b}, —CONHCONR^{a}R^{b}, —NR^{a}R^{b}, —NHCOR^{a}, —NR^{b}COR^{a}, —CSOH, —CSR^{a}, —CSOR^{a}, —CSNR^{a}R^{b}, —CSNHCSNR^{a}R^{b}, —SH, —SR^{a}, —S(C═O)R^{a}, —S(C═O)OR^{a}, —S(C═O)NR^{a}R^{b};
R₅ is selected from the group consisting of hydrogen, C₁₋₉ alkyl, C₂₋₉ alkenyl, C₂₋₉ alkynyl, aryl, heterocyclic, —R^{a}CO, —R^{a}NHCO, and —R^{a}OCO; and
R₆, R^{a}, R^{b}, and R^{c} are independently selected from the group consisting of hydrogen, hydroxyl, amine, C₁₋₉ alkyl, C₂₋₉ alkenyl, C₂₋₉ alkynyl, aryl, and heterocyclic,
   or a crystalline form, hydrate, or pharmaceutically acceptable salt thereof. In another embodiment, the invention provides a method for treating autoimmune diseases in a subject comprising administering one or more of the compounds recited above, or a crystalline form, hydrate, or pharmaceutically acceptable salt thereof, to a patient in need of such treatment.

Another embodiment of the present invention is a method for treating autoimmune diseases in a subject. The method comprises administering to the subject an effective amount of a compound selected from the group consisting of the compounds in Table 1, and crystalline forms, hydrates, or pharmaceutically acceptable salts thereof.

A further embodiment of the present invention is a method for treating autoimmune diseases in a subject. The method comprises administering to the subject an effective amount of a compound selected from the group consisting of:

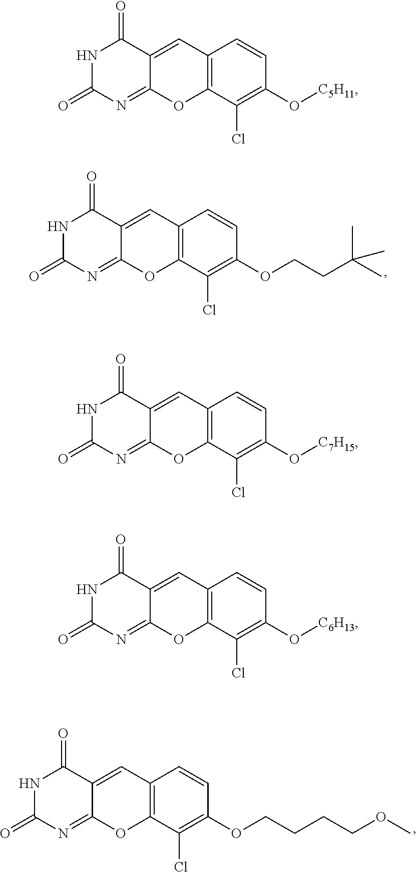

and crystalline forms, hydrates, or pharmaceutically acceptable salts thereof.

An additional embodiment of the present invention is a method for treating rheumatoid arthritis, multiple sclerosis or systemic lupus erythematosus in a subject. The method comprises administering to the subject an effective amount of a compound having the structure of formula (I):

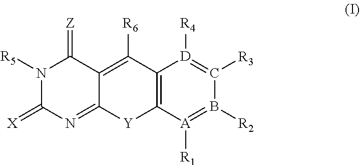

wherein:
A, B, C, and D are independently selected from the group consisting of carbon and nitrogen;
X, Y, and Z are independently selected from the group consisting of oxygen, sulfur, and NR^{a};
R₁, R₂, R₃, and R₄ are independently selected from the group consisting of no atom, hydrogen, halogen, C₁₋₉ alkyl, C₂₋₉ alkenyl, C₂₋₉ alkynyl, aryl, heterocyclic, —OH, —OR^{a}, —OR^{a}OR^{b}, —OR^{a}OR^{b}OR^{c}, —OR^{a}(C═O)R^{b}—O(C═O)R^{a}, —O(C═O)OR^{a}, —O(C═O)NR^{a}R^{b}, cyano, nitro, —CF₃, —CHF₂, —CH₂F, —CHO, —COOH, —COR^{a}, —COOR^{a}, —CONR^{a}R^{b}, —CONHCONR^{a}R^{b}, —NR^{a}R^{b}, —NHCOR^{a}, —NR^{b}COR^{a}, —CSOH, —CSR^{a}, —CSOR^{a}, —CSNR^{a}R^{b}, —CSNHCSNR^{a}R^{b}, —SH, —SR^{a}, —S(C═O)R^{a}, —S(C═O)OR^{a}, —S(C═O)NR^{a}R^{b};
R₅ is selected from the group consisting of hydrogen, C₁₋₉ alkyl, C₂₋₉ alkenyl, C2-9 alkynyl, aryl, heterocyclic, —R^{a}CO, —R^{a}NHCO, and —R^{a}OCO; and
R₆, R^{a}, R^{b}, and R^{c} are independently selected from the group consisting of hydrogen, hydroxyl, amine, C₁₋₉ alkyl, C₂₋₉ alkenyl, C₂₋₉ alkynyl, aryl, and heterocyclic,
or a crystalline form, hydrate, or pharmaceutically acceptable salt thereof.

Another embodiment of the present invention is a method for treating rheumatoid arthritis, multiple sclerosis or systemic lupus erythematosus in a subject. The method comprises administering to the subject an effective amount of a compound selected from the group consisting of the compounds in Table 1, and crystalline forms, hydrates, or pharmaceutically acceptable salts thereof. In another embodiment, the method comprises administering to the subject an effective amount of one or more compounds selected from the group consisting of the compounds in Table 1, and crystalline forms, hydrates, or pharmaceutically acceptable salts thereof.

A further embodiment of the present invention is a method for treating rheumatoid arthritis, multiple sclerosis or systemic lupus erythematosus in a subject. The method comprises administering to the subject an effective amount of a compound selected from the group consisting of:

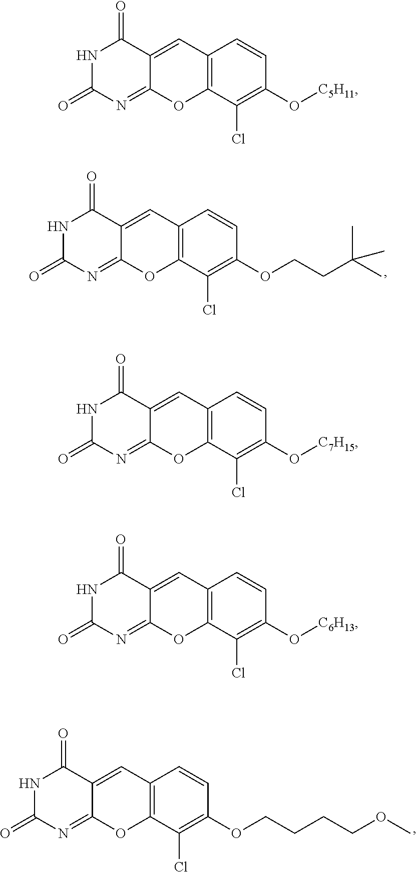

and crystalline forms, hydrates, or pharmaceutically acceptable salts thereof. In another embodiment, the method comprises administering to the subject an effective amount of one or more of the compounds of the above-recited group, or a crystalline form, hydrate, or pharmaceutically acceptable salt thereof. In an embodiment of the methods for treating in a subject described herein, the subject is a mammal, e.g. a human.

In another aspect, the present invention provides the use of any one of the compounds described herein, or of a combination of two or more of the compounds described herein, in the manufacture of a medicament for the treatment of an autoimmune disease. In another aspect, the present invention provides the use of any one of the compounds described herein, or of a combination of two or more of the compounds described herein, or of any composition comprising any one or more of the compounds described herein, to treat an autoimmune disease. In another aspect, the present invention provides any one or more of the compounds or compositions described herein for use in treating an autoimmune disease. In embodiments of any of these aspects, an effective amount of the compound or composition, respectively, is used. In embodiments of any one of these aspects, the compound is selected from the group consisting of the compounds in Table 1, and crystalline forms, hydrates, or pharmaceutically acceptable salts thereof. In an embodiment, the compound is selected from the group consisting of:

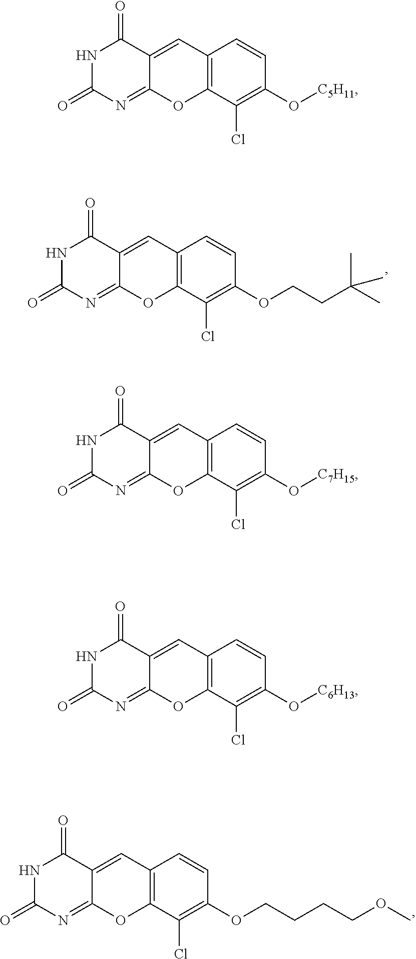

and crystalline forms, hydrates, or pharmaceutically acceptable salts thereof.

In embodiments of any of these aspects, the autoimmune disease is any of rheumatoid arthritis, multiple sclerosis, Systemic lupus erythematosus, Type I diabetes, Ankylosing spondylitis, Spondyloarthropathies, Crohn's disease (inflammatory bowel disease), Grave's disease, Hashimoto's thyroiditis, Myasthenia gravis, Psoriasis, Lymphoproliferative disease (ALPS), Sjogren's syndrome, Autoimmune neuropathies, Gullian-Barre syndrome, Autoimmune uveitis, Autoimmune hemolytic anemia, Pernicious anemia, Aplastic anemia, Pure red cell anemia, Autoimmune thrombocytopenia, Temporal arteritis, Anti-phospholipid syndrome, Vasculitides, Wegener's granulomatosis, Behcet's disease, Dermatitis herpetiformis, Pemphigus vulgaris, Vitiligo, Primary biliary cirrhosis, Autoimmune hepatitis, Autoimmune oophoritis and orchitis, Autoimmune disease of the adrenal gland, Scleroderma, Polymyositis, Dermatomyositis, Autoimmune menagitis, Autoimmune dermatitis, Alopecia areata, Autoimmune uveitis, Allergic encephalomyelitis, Interstitial lung fibrosis, Seronegative arthropathies, Sarcoidosis, Orchitis/vasectomy reversal procedure, Raynoud's disease, Type B insulin-resistant diabetes, Antibody-mediated cytotoxicity, Type III hypersensitivity reactions, POEMS syndrome, Polyneuropathy, Organomegaly, Endocrinopathy, Monoclonal gammopathy, Skin changes syndrome, Pemphigus, Mixed connective tissue diseases, Idiopathic Addison's disease, Post-MI cardiotomy syndrome, Wilson's disease, Hemachromatosis, Alpha-1-antitrypsin deficiency, Osteoporosis, Hypothalamic-pituitary-adrenal axis evaluation, Familial hematophagocytic lymphohistiocytosis, Pre-eclampsia, OKT3 therapy, Anti-CD3 therapy, Cytokine therapy, Chemotherapy, Radiation therapy and Immune tolerance therapy via co-administration of self-antigens or self-tissues. In an embodiment, the condition is rheumatoid arthritis, multiple sclerosis or systemic lupus erythematosus.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1D: FP assay optimization. FIG. 1A shows a line graph of results from 10 nM CD28RE FITC fluorescence polarization assays. c-Rel(281) at 2-fold dilutions (2000 to 15.625 nM) were mixed with CD28RE-FITC (10, 3.3, 1.1, 0.33, 0.11 nM) in the FP buffer for 30 minutes. mP values were used to calculate Signal/Background ratio. Only the data for 10 nM and 0.33 nM are shown here. FIG. 1B shows a line graph of cold competition with specific (CD28RE) and non-specific (Oct1) oligo. FIG. 1C is a dot plot showing distribution of fluorescence polarization signals in a representative 384-well plate. Z′ value for the plate is 0.83. FIG. 1D shows a line graph of results from 0.33 nM CD28RE FITC fluorescence polarization assays.

FIG. 2 shows a line graph showing Rel/NF-κB inhibition by compound 6 by EMSA (electrophoretic mobility shift assay).

FIG. 3A shows a graph of animal's body weight versus days after treatment of the animal as described in Example 1. FIG. 3B shows a graph of Change in animal's body weight from example 1. A value of 100% indicates no change, i.e., the body weight is 100% of the original body weight.

FIG. 4: Rheumatoid Arthritis clinical score from example 1.

FIG. 5A: Animal paw volume from example 1. FIG. 5B: Change in animal paw volume from example 1.

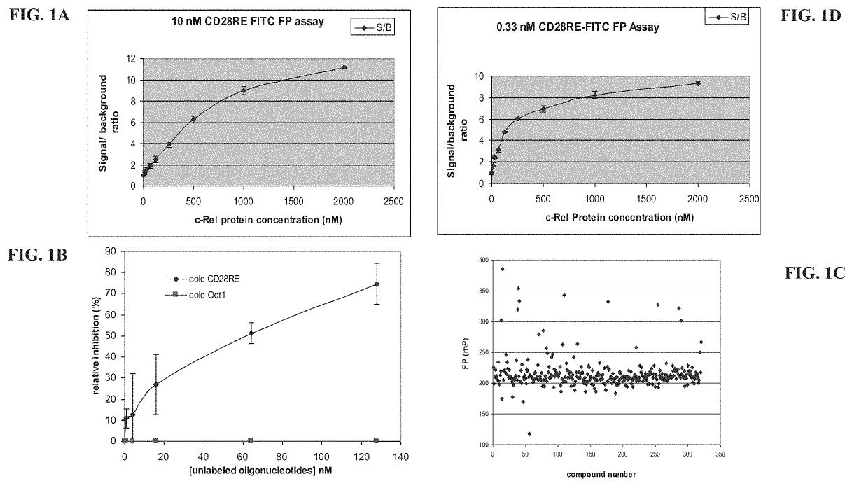

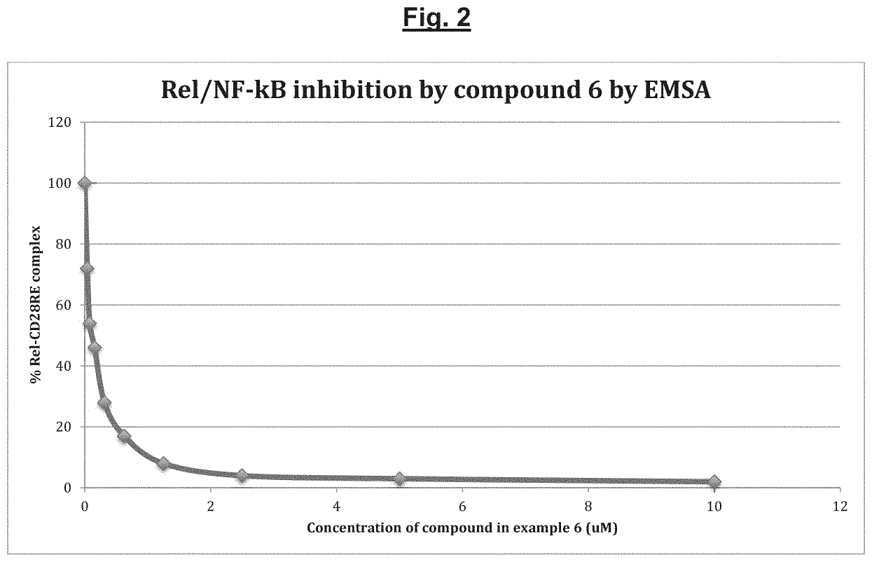

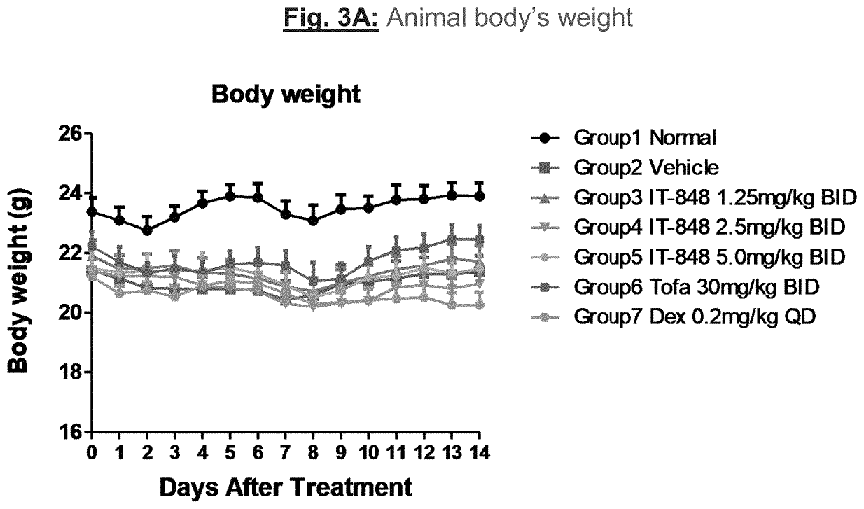

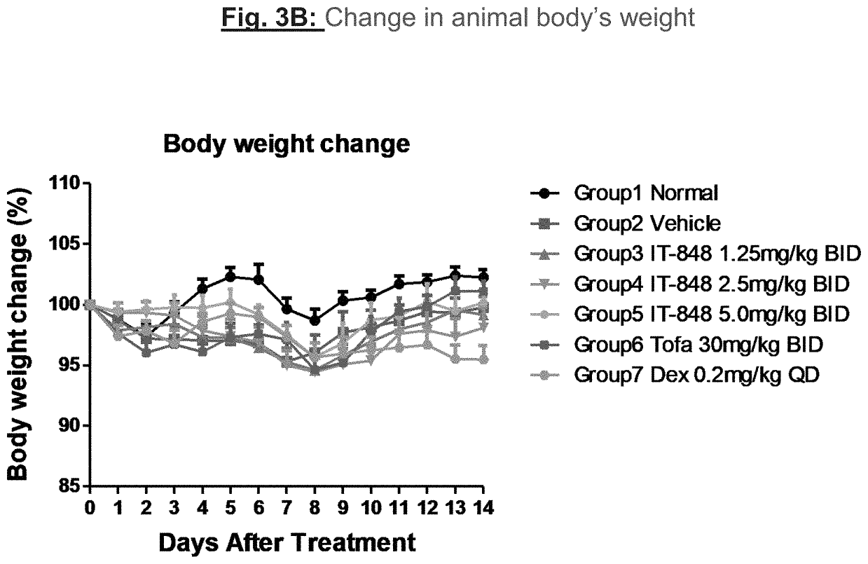

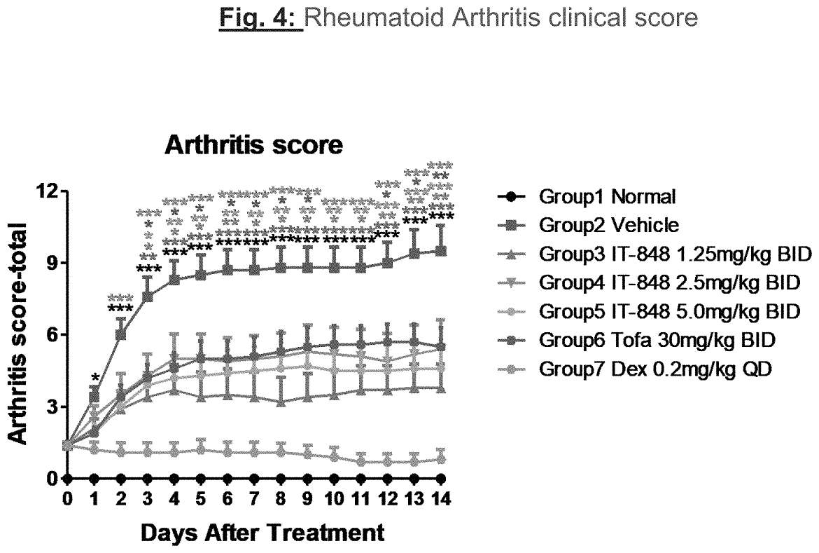

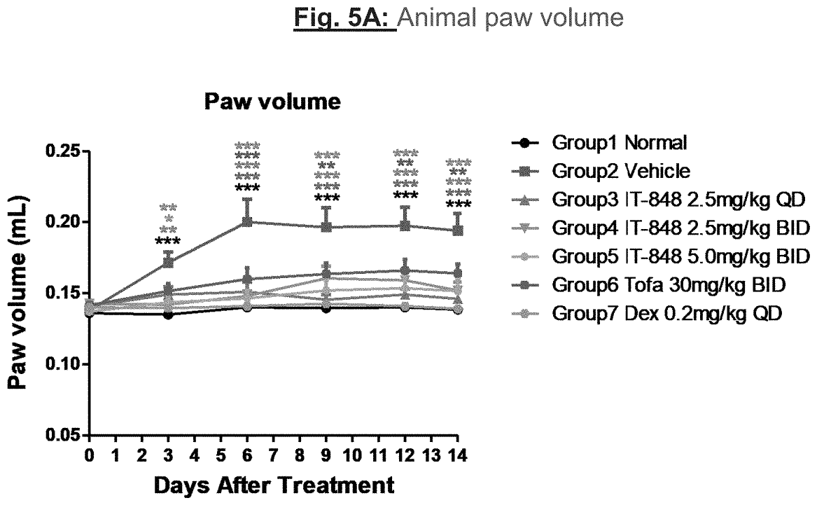

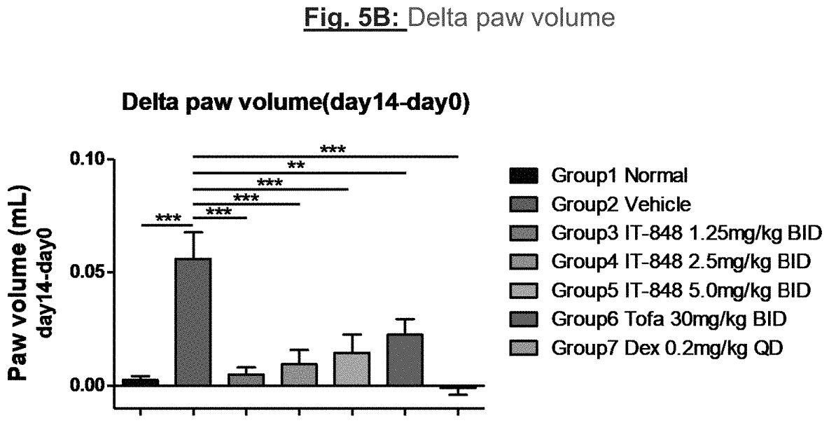

### DETAILED DESCRIPTION OF THE INVENTION

The terminology used herein is for the purpose of describing particular embodiments only and is not intended to be limiting. As used in the specification and the appended claims, the singular forms “a,” “an,” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

The term “about” generally indicates within ±0.5%, 1%, 2%, 5%, or up to ±10% of the indicated value. For example, an amount of “about 10 wt %” generally indicates, in its broadest sense, 10 wt %±10%, which indicates 9.0-11.0 wt %. The term “about” may alternatively indicate a variation or average in a physical characteristic of a group.

One embodiment of the present invention is a compound. The compound has the structure of formula (I).

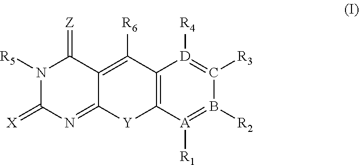

wherein:
A, B, C, and D are independently selected from the group consisting of carbon and nitrogen;
X, Y, and Z are independently selected from the group consisting of oxygen, sulfur, and NR^{a};
R₁, R₂, R₃, and R₄ are independently selected from the group consisting of no atom, hydrogen, halogen, C₁₋₉ alkyl, C₂₋₉ alkenyl, C₂₋₉ alkynyl, aryl, heterocyclic, —OH, —OR^{a}, —OR^{a}OR^{b}, —OR^{a}OR^{b}R^{c}, —OR^{a}(C═O)R^{b}, —O(C═O)R^{a}, —O(C═O)OR^{a}, —O(C═O)NR^{a}R^{b}, cyano, nitro, —CF₃, —CHF₂, —CH₂F, —CHO, —COOH, —COR^{a}, —COOR^{a}, —CONR^{a}R^{b}, —CONHCONR^{a}R^{b}, —NR^{a}R^{b}, —NHCOR^{a}, —NR^{b}COR^{a}, —CSOH, —CSR^{a}, —CSOR^{a}, —CSNR^{a}R^{b}, —CSNHCSNR^{a}R^{b}, —SH, —SR^{a}, —S(C═O)R^{a}, —S(C═O)OR^{a}, —S(C═O)NR^{a}R^{b};
R₅ is selected from the group consisting of hydrogen, C₁₋₉ alkyl, C₂₋₉ alkenyl, C₂₋₉ alkynyl, aryl, heterocyclic, —R^{a}CO, —R^{a}NHCO, and —R^{a}OCO; and
R₆, R^{a}, R^{b}, and R^{c} are independently selected from the group consisting of hydrogen, hydroxyl, amine, C₁₋₉ alkyl, C₂₋₉ alkenyl, C₂₋₉ alkynyl, aryl, and heterocyclic, or is a crystalline form, hydrate, or pharmaceutically acceptable salt thereof.

As used herein, the term “compound” refers to two or more atoms that are connected by one or more chemical bonds. In the present invention, “chemical bonds” and “bonds” are interchangeable and include, but are not limited to, covalent bonds, ionic bonds, hydrogen bonds, and van der Waals interactions. Covalent bonds of the present invention include single, double, and triple bonds. Compounds of the present invention include, but are not limited to, organic molecules. Atoms that comprise the compounds of the present invention are “linked” if they are connected by a chemical bond of the present invention.

Organic compounds of the present invention include linear, branched, and cyclic hydrocarbons with or without functional groups. The term “C_{x-y}” when used in conjunction with a chemical moiety, such as, alkyl, alkenyl, alkynyl or alkoxy is meant to include groups that contain from x to y carbons in the chain. For example, the term “C_{x-y} alkyl” means substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. The terms “C_{x-y} alkenyl” and “C_{x-y} alkynyl” refer to substituted or unsubstituted unsaturated aliphatic groups analogous in length and possible substitution to the alkyls described above, but containing at least one double or triple bond respectively.

The term “independently selected” and grammatical variations thereof mean that, in a chemical structure of the present invention, (e.g., formula I), if more than one atom in the structure can be selected from a list of elements, those atoms may or may not be of the same element. Similarly, if more than one chemical moiety in the structure can be selected from a list of chemical moieties, those moieties may or may not be the same.

In one aspect of this embodiment,

X, Y, and Z are independently selected from the group consisting of oxygen and sulfur;
R₁ is selected from the group consisting of —H, —F, —Cl, —OMe, and —OEt;
R₂ is selected from the group consisting of —H, —CH₃, —OH, —OMe, —OEt, -Me, -Et, -nPr, —O-nPr, —OEtnPr, —OC₄H₉, —OC₅H₁₁, —OC₆H₁₃, —OC₇H₁₅, —O-isobutyl, —O-isopentyl, —OCₙH₂ₙOMe, —OCₙH₂ₙOCₘH₂ₘOMe, —OCₙH₂ₙOH, —OCₙH₂ₙOCₘH₂ₘOH, OCₙH₂ₙOEt, —OCₙH₂ₙOCₘH₂ₘOEt, —O—CₙH₂ₙCOOH, —O—CₙH₂ₙCONH₂, —O—CₙH₂ₙCONHMe,

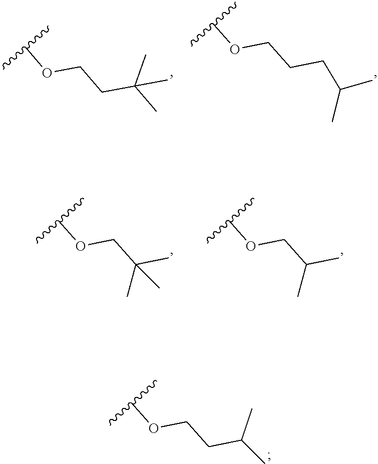

R₃ is selected from the group consisting of —H, —Cl, —Br, —F, and —OMe;
R₄ is selected from the group consisting of —H and —OMe;
R₅ is selected from the group consisting of —H, -Me, -Et, —Pr, -iPr, -Ph, iBu and -nBu;
R₆ is selected from the group consisting of —H and —CH₃;
m is 2, 3, 4 or 5; and, n is 2, 3, 4, or 5.

In another aspect of this embodiment,

X, Y, and Z are independently selected from the group consisting of oxygen and sulfur; and
R₆ is hydrogen.

In an aspect of this embodiment,

Z is oxygen;
R₁ and R₃ are selected from the group consisting of hydrogen, halogen, —CN, and —CF₃;
R₂ is selected from the group consisting of C₁₋₉ alkoxy, —OCₙH₂ₙOMe, —OCₙH₂ₙOCₘH₂ₘOMe, —OCₙH₂ₙOH, —OCₙH₂ₙOCₘH₂ₘOH, OCₙH₂ₙOEt, —OCₙH₂ₙOCₘH₂ₘOEt, —O—CₙH₂ₙCOOH, —O—CₙH₂ₙCONH₂, —O—CₙH₂ₙCONHMe, and —OH;
R₄ is selected from the group consisting of hydrogen, C₁₋₉ alkoxy and —OH; m is 2, 3, 4 or 5; and n is 2, 3, 4, or 5.

In an aspect of this embodiment,

X and Y are oxygen; and
R₄ is hydrogen.

In an aspect of this embodiment, the compound is selected from the group consisting of:

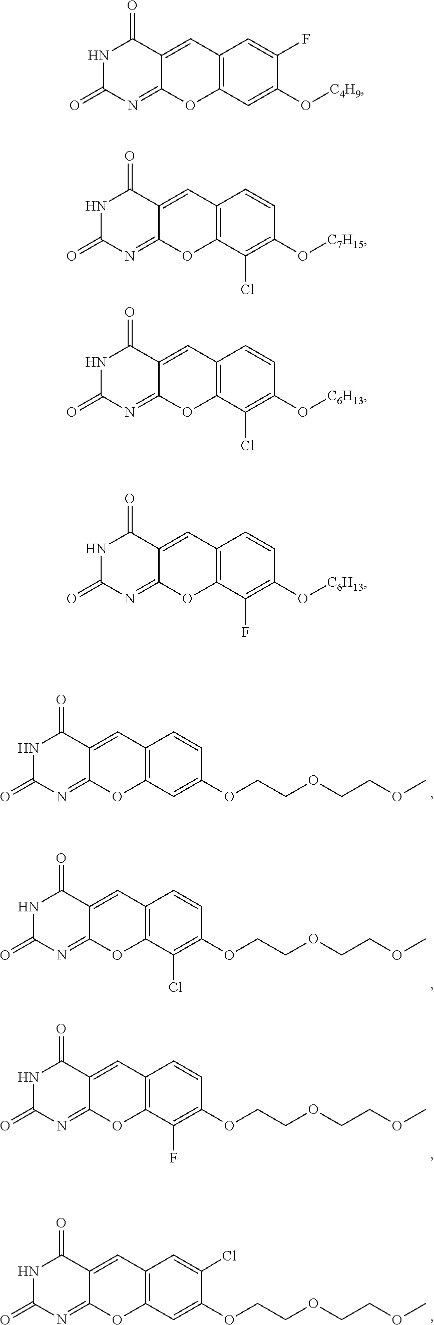

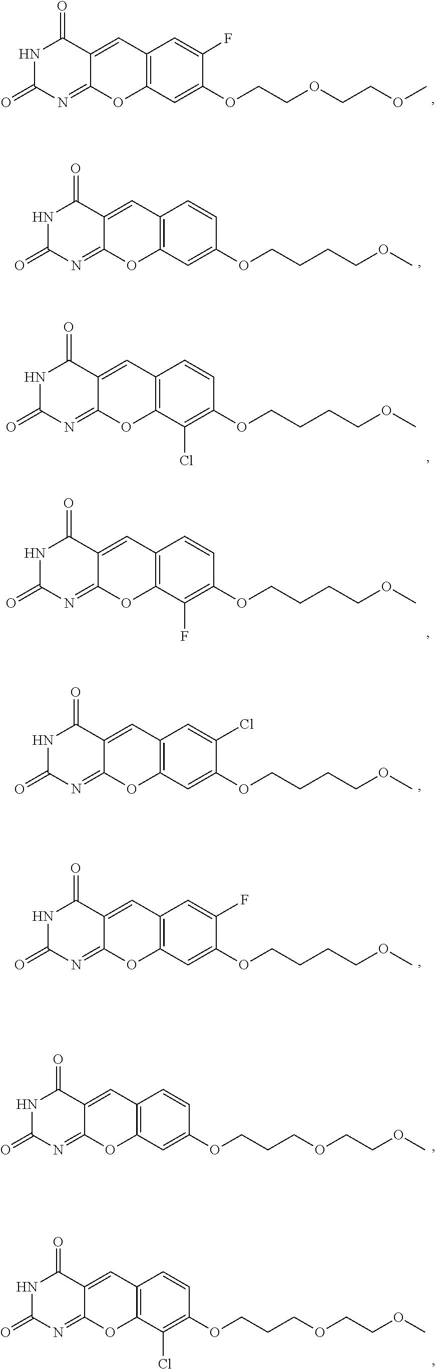

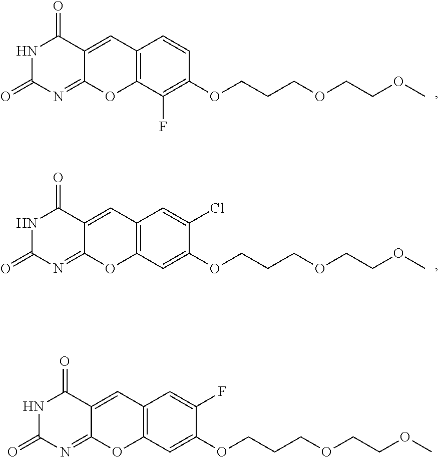 [SMILES: COCCOCCCOc1ccc2cc3c(=O)[nH]c(=O)nc-3oc2c1F ; COCCOCCCOc1cc2oc3nc(=O)[nH]c(=O)c-3cc2cc1Cl ; COCCOCCCOc1cc2oc3nc(=O)[nH]c(=O)c-3cc2cc1F]

and crystalline forms, hydrates, or pharmaceutically acceptable salts thereof.

In an additional aspect of this embodiment, R₁ and R₂ are linked by at least one bond.

In a further aspect of this embodiment, R₂ and R₃ are linked by at least one bond.

In a further aspect of this embodiment, R₃ and R₄ are linked by at least one bond.

Another embodiment of the present invention is a compound. The compound is selected from the group consisting of the compounds in Table 1, and crystalline forms, hydrates, or pharmaceutically acceptable salts thereof.

A further embodiment of the present invention is a compound. The compound is selected from the group consisting of:

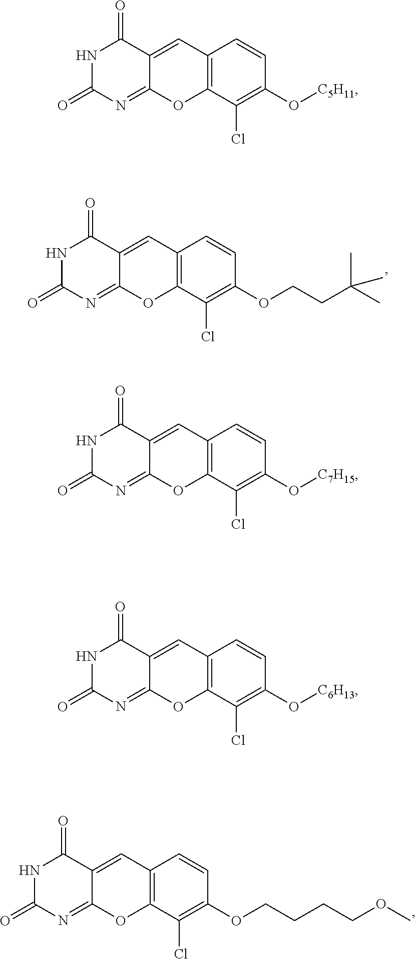

and crystalline forms, hydrates, or pharmaceutically acceptable salts thereof.

An additional embodiment of the present invention is a compound capable of inhibiting NF-κB. The compound has the structure of formula (I):

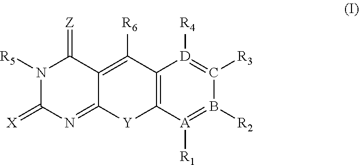

wherein:
A, B, C, and D are independently selected from the group consisting of carbon and nitrogen;
X, Y, and Z are independently selected from the group consisting of oxygen, sulfur, and NR^{a};
R₁, R₂, R₃, and R₄ are independently selected from the group consisting of no atom, hydrogen, halogen, C₁₋₉ alkyl, C₂₋₉ alkenyl, C₂₋₉ alkynyl, aryl, heterocyclic, —OH, —OR^{a}, —OR^{a}OR^{b}, —OR^{a}OR^{b}OR^{c}, —O(C═O)R^{a}, —O(C═O)OR^{a}, —O(C═O)NR^{a}R^{b}, cyano, nitro, CF₃, CHF₂, CH₂F, —CHO, —COOH, —COR^{a}, —COOR^{a}, —CONR^{a}R^{b}, —CONHCONR^{a}R^{b}, —NR^{a}R^{b}, —NHCOR^{a}, —NR^{b}COR^{a}, —CSOH, —CSR^{a}, —CSOR^{a}, —CSNR^{a}R^{b}, —CSNHCSNR^{a}R^{b}, —SH, —SR^{a}, —S(C═O)R^{a}, —S(C═O)OR^{a}, —S(C═O)NR^{a}R^{b};
R₅ is selected from the group consisting of hydrogen, C₁₋₉ alkyl, C₂₋₉ alkenyl, C2-9 alkynyl, aryl, heterocyclic, —R^{a}CO, —R^{a}NHCO, and —R^{a}OCO; and
R₆, R^{a}, R^{b}, and R^{c} are independently selected from the group consisting of hydrogen, hydroxyl, amine, C₁₋₉ alkyl, C₂₋₉ alkenyl, C₂₋₉ alkynyl, aryl, and heterocyclic, or is a crystalline form, hydrate, or pharmaceutically acceptable salt thereof.

In the present invention, the compound capable of inhibiting NF-κB may function as a direct or indirect NF-κB/Rel inhibitor. A direct NF-κB/Rel inhibitor is a compound that binds to or interacts with NF-κB/Rel directly and inhibits its DNA binding and transcriptional function. An indirect NF-κB/Rel inhibitor is a compound that binds to or interacts with a compound other than NF-κB/Rel, thereby generating a downstream inhibitory effect on NF-κB/Rel activity.

In one aspect of this embodiment,

X, Y, and Z are independently selected from the group consisting of oxygen and sulfur;
R₁ is selected from the group consisting of —H, —F, —Cl, —OMe, and —OEt;
R₂ is selected from the group consisting of —H, —CH₃, —OH, —OMe, —OEt, -Et, -nPr, —O-nPr, —OEtnPr, —OC₄H₉, —OC₅H₁₁, —OC₆H₁₃, —OC₇H₁₅, —O-isobutyl, —O-isopentyl, —OCₙH₂ₙOMe, —OCₙH₂ₙOCₘH₂ₘOMe, —OCₙH₂ₙOH, —OCₙH₂ₙOCₘH₂ₘOH, OCₙH₂ₙOEt, —OCₙH₂ₙOCₘH₂ₘOEt, —O—CₙH₂ₙCOOH, —O—CₙH₂ₙCONH₂, —O—CₙH₂ₙCONHMe,

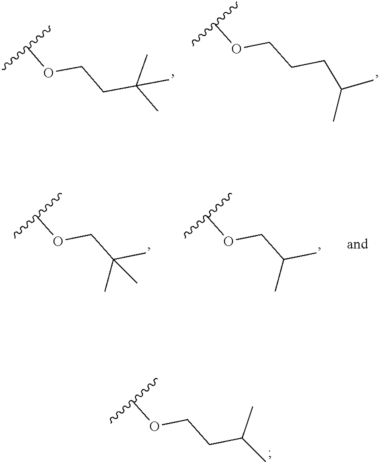

R₃ is selected from the group consisting of —H, —Cl, —Br, —F, and —OMe;
R₄ is selected from the group consisting of —H and —OMe;
R₅ is selected from the group consisting of —H, -Me, and -nBu;
R₆ is selected from the group consisting of —H and —CH₃;
m is 2, 3, 4 or 5; and n is 2, 3, 4, or 5.

In another aspect of this embodiment,

X, Y, and Z are independently selected from the group consisting of oxygen and sulfur; and
R₆ is hydrogen.

In an aspect of this embodiment,

Z is oxygen;
R₁ and R₃ are selected from the group consisting of hydrogen, halogen, —CN, and —CF₃;
R₂ is selected from the group consisting of C₁₋₉ alkoxy, —OCₙH₂ₙOMe, —OCₙH₂ₙOCₘH₂ₘOMe, —OCₙH₂ₙOH, —OCₙH₂ₙOCₘH₂ₘOH, —OCₙH₂ₙOEt, —OCₙH₂ₙOCₘH₂ₘOEt, and —OH;
R₄ is selected from the group consisting of hydrogen, C₁₋₉ alkoxy and —OH;
m is 2, 3, 4 or 5; and n is 2, 3, 4, or 5.

In an aspect of this embodiment,

X and Y are oxygen; and
R₄ is hydrogen.

In an aspect of this embodiment, the compound is selected from the group consisting of:

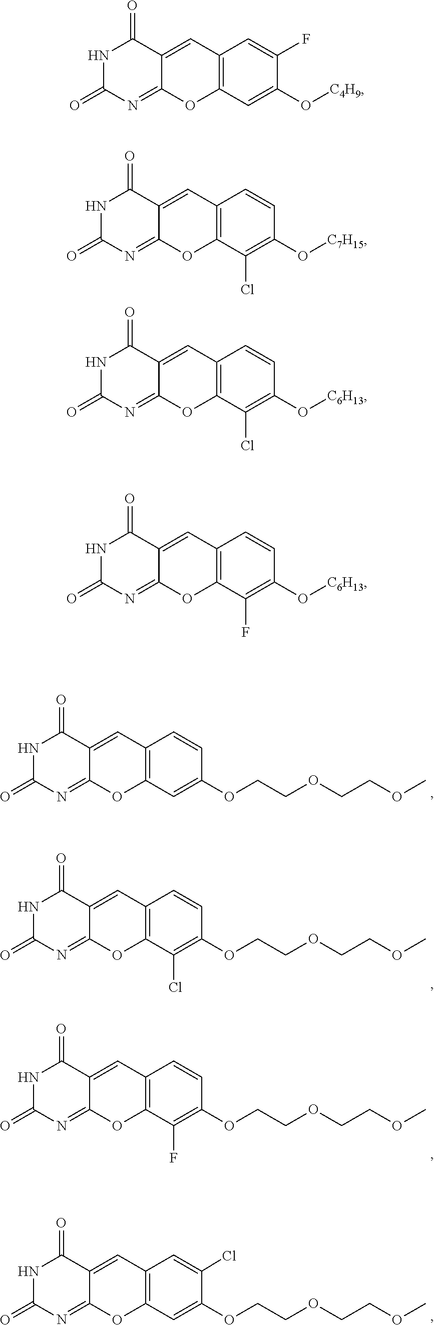

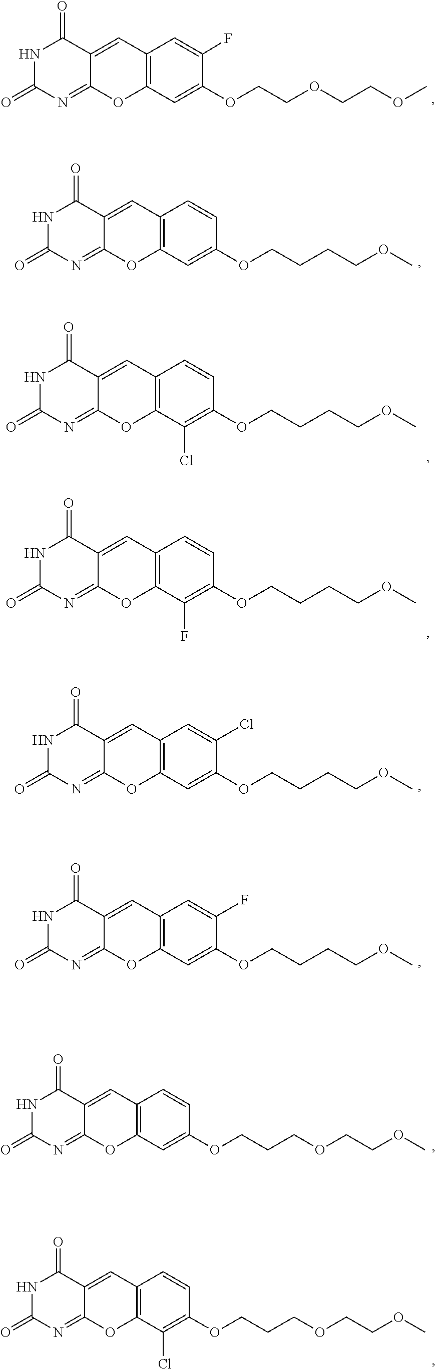

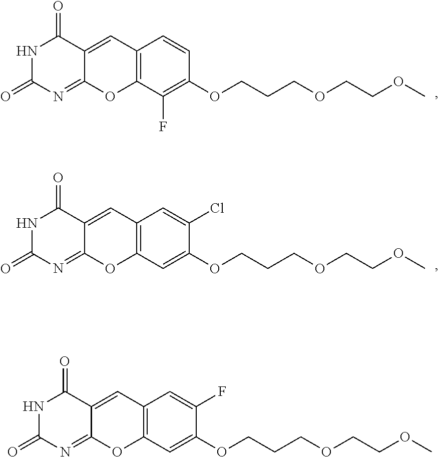 [SMILES: COCCOCCCOc1ccc2cc3c(=O)[nH]c(=O)nc-3oc2c1F ; COCCOCCCOc1cc2oc3nc(=O)[nH]c(=O)c-3cc2cc1Cl ; COCCOCCCOc1cc2oc3nc(=O)[nH]c(=O)c-3cc2cc1F]

and crystalline forms, hydrates, or pharmaceutically acceptable salts thereof.

In an additional aspect of this embodiment, R₁ and R₂ are linked by at least one bond.

In a further aspect of this embodiment, R₂ and R₃ are linked by a bond.

In another aspect of this embodiment, R₃ and R₄ are linked by a bond.

A further embodiment of the present invention is a compound capable of inhibiting NF-κB. The compound is selected from the group consisting of the compounds in Table 1, and crystalline forms, hydrates, or pharmaceutically acceptable salts thereof.

An additional embodiment of the present invention is a compound capable of inhibiting NF-κB. The compound is selected from the group consisting of:

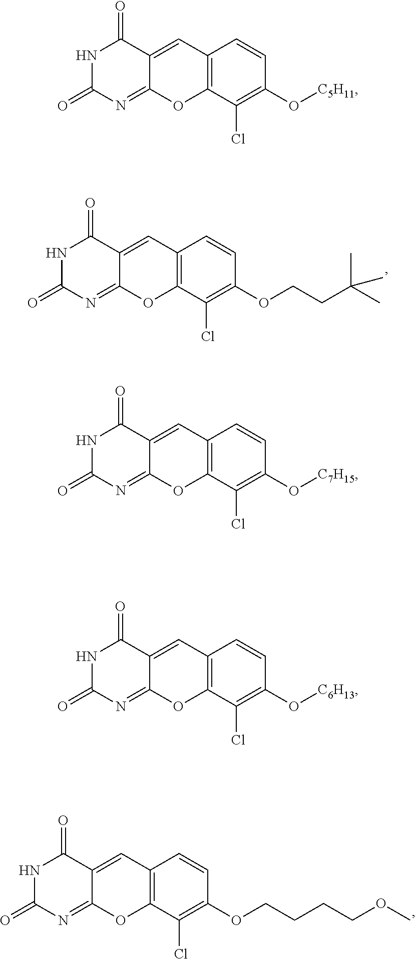

and crystalline forms, hydrates, or pharmaceutically acceptable salts thereof.

R₁ and R₂, R₂ and R₃, and R₃ and R₄, can be joined to form a ring, aromatic or not. The ring can be a hydrocarbon ring or a heterocyclic ring.

Another embodiment of the present invention is a pharmaceutical composition. The pharmaceutical composition comprises a pharmaceutically acceptable carrier and any of the compounds disclosed herein. The pharmaceutical composition may also include any number of other auxiliary agents used in the art, e.g., buffering agents, stabilizing agents, emulsifying agents, pH adjusting agents, surfactants, and flavorants.

A further embodiment of the present invention is a method of inhibiting NF-κB in a cell. The method comprises contacting the cell with any of the compounds disclosed herein.

As used herein, the term “contacting” means bringing a compound of the present invention into close proximity to the cells of the present invention. This may be accomplished using conventional techniques of drug delivery to mammals (e.g., tail vein injection, intravenous injection, peroral administration) or in the in vitro situation by, e.g., providing a compound of the present invention to a culture media to which the cells of the present invention are exposed.

Cells of the present invention include any cell type, cancerous or non-cancerous, in vitro or in vivo, that expresses NF-κB or any NF-κB family member. Cells of the present invention include, but are not limited to, human, monkey, ape, hamster, rat, or mouse cells. In some embodiments, cells of the present invention include, but are not limited to, CHO (e.g., CHO K1, DXB-11 CHO, Veggie-CHO), COS (e.g., COS-7), retinal cells, Vero, CV1, kidney (e.g., HEK293, 293 EBNA, MSR293, MDCK, HaK, BHK), HeLa, HepG2, WI38, MRC 5, Col0205, HB 8065, HL-60, (e.g., BHK21), Jurkat, Daudi, A431 (epidermal), CV-1, U937, 3T3, L cells, C127 cells, SP2/0, NS-0, MMT 060562, Sertoli cells, BRL 3A cells, HT1080 cells, myeloma cells, tumor cells, and any cell line derived from any of the aforementioned cells.

An additional embodiment of the present invention is a method for treating autoimmune diseases in a subject. The method comprises administering to the subject an effective amount of a compound having the structure of formula (I):

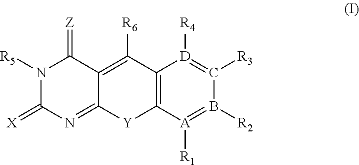

wherein:
A, B, C, and D are independently selected from the group consisting of carbon and nitrogen;
X, Y, and Z are independently selected from the group consisting of oxygen, sulfur, and NR^{a};
R₁, R₂, R₃, and R₄ are independently selected from the group consisting of no atom, hydrogen, halogen, C₁₋₉ alkyl, C₂₋₉ alkenyl, C₂₋₉ alkynyl, aryl, heterocyclic, —OH, —OR^{a}, —OR^{a}OR^{b}, —OR^{a}OR^{b}OR^{c}, —OR^{a}(C═O)R^{b}—O(C═O)R^{a}, —O(C═O)OR^{a}, —O(C═O)NR^{a}R^{b}, cyano, nitro, —CF₃, —CHF₂, —CH₂F, —CHO, —COOH, —COR^{a}, —COOR^{a}, —CONR^{a}R^{b}, —CONHCONR^{a}R^{b}, —NR^{a}R^{b}, —NHCOR^{a}, —NR^{b}COR^{a}, —CSOH, —CSR^{a}, —CSOR^{a}, —CSNR^{a}R^{b}, —CSNHCSNR^{a}R^{b}, —SH, —SR^{a}, —S(C═O)R^{a}, —S(C═O)OR^{a}, —S(C═O)NR^{a}R^{b};
R₅ is selected from the group consisting of hydrogen, C₁₋₉ alkyl, C₂₋₉ alkenyl, C2-9 alkynyl, aryl, heterocyclic, —R^{a}CO, —R^{a}NHCO, and —R^{a}OCO; and
R₆, R^{a}, R^{b}, and R^{c} are independently selected from the group consisting of hydrogen, hydroxyl, amine, C₁₋₉ alkyl, C₂₋₉ alkenyl, C₂₋₉ alkynyl, aryl, and heterocyclic, or a crystalline form, hydrate, or pharmaceutically acceptable salt thereof.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present invention may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population may fail to respond or respond inadequately to treatment.

Autoimmune diseases include rheumatoid arthritis, multiple sclerosis, Systemic lupus erythematosus, Type I diabetes, Ankylosing spondylitis, Spondyloarthropathies, Crohn's disease (inflammatory bowel disease), Grave's disease, Hashimoto's thyroiditis, Myasthenia gravis, Psoriasis, Lymphoproliferative disease (ALPS), Sjogren's syndrome, Autoimmune neuropathies, Gullian-Barre syndrome, Autoimmune uveitis, Autoimmune hemolytic anemia, Pernicious anemia, Aplastic anemia, Pure red cell anemia, Autoimmune thrombocytopenia, Temporal arteritis, Anti-phospholipid syndrome, Vasculitides, Wegener's granulomatosis, Behcet's disease, Dermatitis herpetiformis, Pemphigus vulgaris, Vitiligo, Primary biliary cirrhosis, Autoimmune hepatitis, Autoimmune oophoritis and orchitis, Autoimmune disease of the adrenal gland, Scleroderma, Polymyositis, Dermatomyositis, Autoimmune menagitis, Autoimmune dermatitis, Alopecia areata, Autoimmune uveitis, Allergic encephalomyelitis, Interstitial lung fibrosis, Seronegative arthropathies, Sarcoidosis, Orchitis/vasectomy reversal procedure, Raynoud's disease, Type B insulin-resistant diabetes, Antibody-mediated cytotoxicity, Type III hypersensitivity reactions, POEMS syndrome, Polyneuropathy, Organomegaly, Endocrinopathy, Monoclonal gammopathy, Skin changes syndrome, Pemphigus, Mixed connective tissue diseases, Idiopathic Addison's disease, Post-MI cardiotomy syndrome, Wilson's disease, Hemachromatosis, Alpha-1-antitrypsin deficiency, Osteoporosis, Hypothalamic-pituitary-adrenal axis evaluation, Familial hematophagocytic lymphohistiocytosis, Pre-eclampsia, OKT3 therapy, Anti-CD3 therapy, Cytokine therapy, Chemotherapy, Radiation therapy and Immune tolerance therapy via co-administration of self-antigens or self-tissues.

As used herein, a “subject” is a mammal, preferably, a human. In addition to humans, categories of mammals within the scope of the present invention include, for example, farm animals, domestic animals, laboratory animals, etc. Some examples of farm animals include cows, pigs, horses, goats, etc. Some examples of domestic animals include dogs, cats, etc. Some examples of laboratory animals include primates, rats, mice, rabbits, guinea pigs, etc.

The NF-κB inhibitors of the present invention or compositions containing the same of the present invention may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, the NF-κB inhibitors or compositions containing same of the present invention may be administered in conjunction with other treatments. The NF-κB inhibitors or the compositions of the present invention may be encapsulated or otherwise protected against gastric or other secretions, if desired.

In the present invention, an “effective amount” or a “therapeutically effective amount” of the NF-κB inhibitors of the present invention, including the compositions containing same, as well as chemotherapeutic agents and targeted therapeutic agents of the present invention is an amount of such inhibitor, composition, or agent that is sufficient to induce beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of an agent or composition according to the invention will be that amount of the agent or composition, which is the lowest dose effective to produce the desired effect. The effective dose of an agent or composition of the present invention may be administered as two, three, four, five, six or more sub-doses, administered separately at appropriate intervals throughout the day.

In one aspect of this embodiment,

X, Y, and Z are independently selected from the group consisting of oxygen and sulfur;
R₁ is selected from the group consisting of —H, —F, —Cl, —OMe, and —OEt;
R₂ is selected from the group consisting of —H, —CH₃, —OH, —OMe, —OEt, -Me, -Et, -nPr, —O-nPr, —OEtnPr, —OC₄H₉, —OC₅H₁₁, —OC₆H₁₃, —OC₇H₁₅, —O-isobutyl, —O-isopentyl, —OCₙH₂ₙOMe, —OCₙH₂ₙOCₘH₂ₘOMe, —OCₙH₂ₙOH, —OCₙH₂ₙOCₘH₂ₘOH, —OCₙH₂ₙOEt, —OCₙH₂ₙOCₘH₂ₘOEt, —O—CₙH₂ₙCOOH, —O—CₙH₂ₙCONH₂, —O—CₙH₂ₙCONHMe,

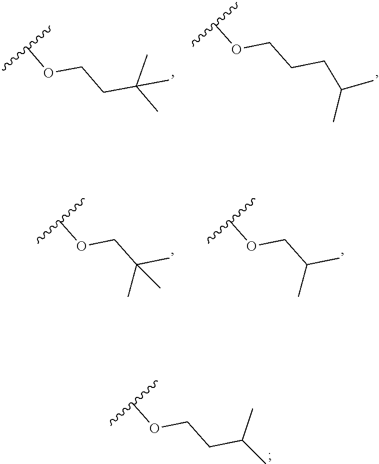

and
R₃ is selected from the group consisting of —H, —Cl, —Br, —F, and —OMe;
R₄ is selected from the group consisting of —H and —OMe;
R₅ is selected from the group consisting of —H, -Me, -Et, —Pr, -iPr, -Ph, -iBu, and -nBu;
R₆ is selected from the group consisting of —H and —CH₃,
m is 2, 3, 4 or 5; and
n is 2, 3, 4, or 5.

In another aspect of this embodiment,

X, Y, and Z are independently selected from the group consisting of oxygen and sulfur; and
R₆ is hydrogen.

In an aspect of this embodiment,

Z is oxygen;
R₁ and R₃ are selected from the group consisting of hydrogen, halogen, —CN, and —CF₃;
R₂ is selected from the group consisting of C₁₋₉ alkoxy, —OCₙH₂ₙOMe, —OCₙH₂ₙOCₘH₂ₘOMe, —OCₙH₂ₙOH, —OCₙH₂ₙOCₘH₂ₘOH, OCₙH₂ₙOEt, —OCₙH₂ₙOCₘH₂ₘOEt, —O—CₙH₂ₙCOOH, —O—CₙH₂ₙCONH₂, —O—CₙH₂ₙCONHMe, and —OH;
m is 2, 3, 4 or 5;
n is 2, 3, 4, or 5; and
R₄ is selected from the group consisting of hydrogen, C₁₋₉ alkoxy and —OH.

In an aspect of this embodiment, X and Y are oxygen; and R₄ is hydrogen.

In an aspect of this embodiment, the compound is selected from the group consisting of:

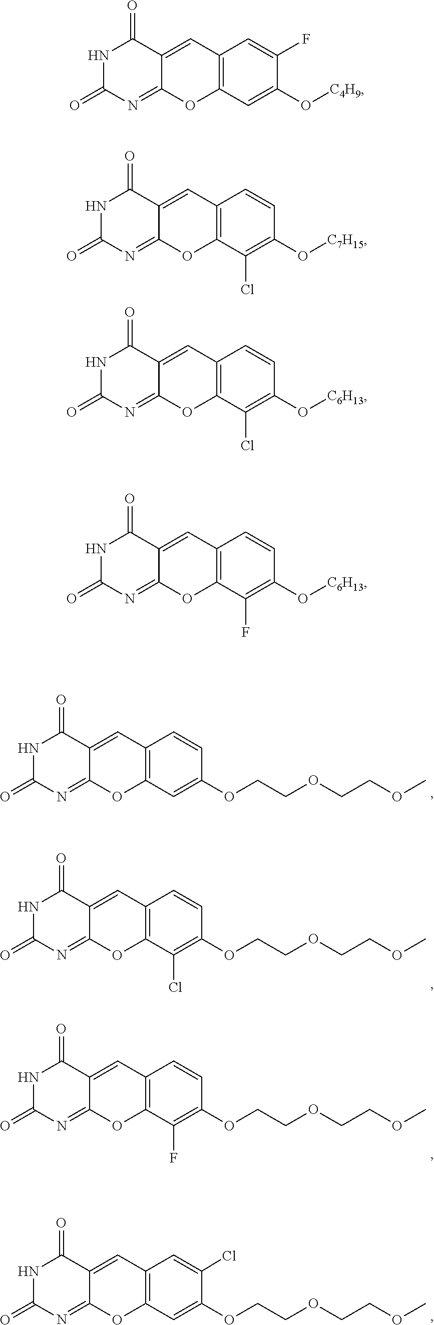

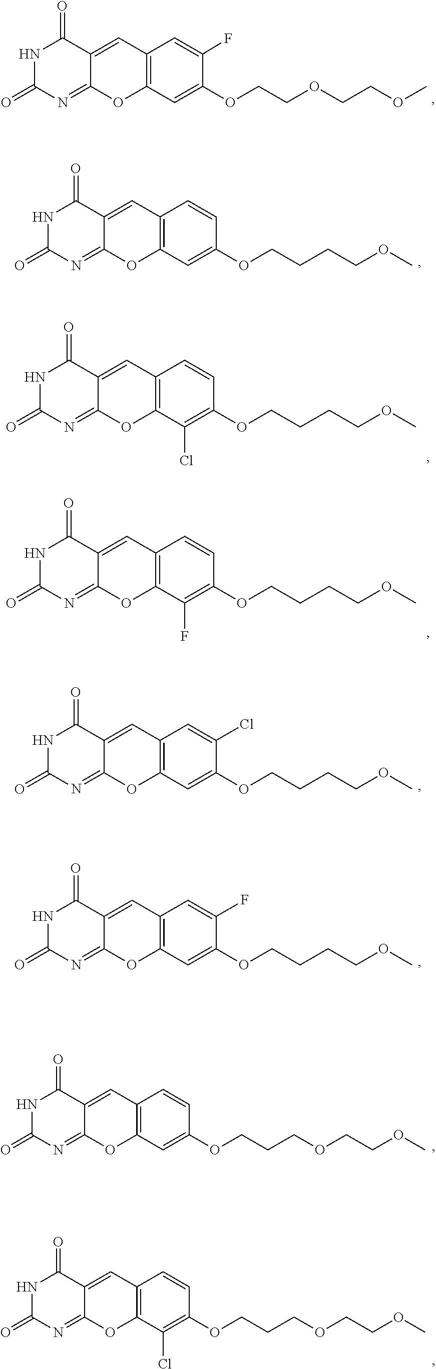

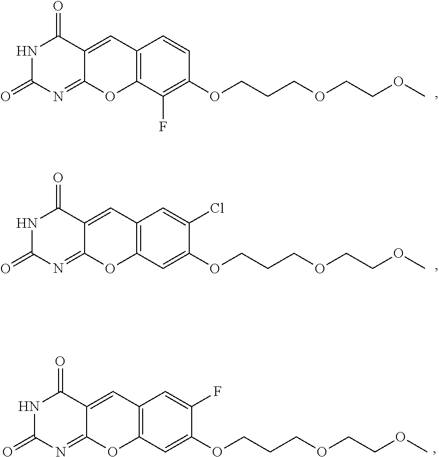 [SMILES: COCCOCCCOc1ccc2cc3c(=O)[nH]c(=O)nc-3oc2c1F ; COCCOCCCOc1cc2oc3nc(=O)[nH]c(=O)c-3cc2cc1Cl ; COCCOCCCOc1cc2oc3nc(=O)[nH]c(=O)c-3cc2cc1F]

and crystalline forms, hydrates, or pharmaceutically acceptable salts thereof.

In another aspect of this embodiment, the autoimmune disease is selected from rheumatoid arthritis, multiple sclerosis, Systemic lupus erythematosus, Type I diabetes, Ankylosing spondylitis, Spondyloarthropathies, Crohn's disease (inflammatory bowel disease), Grave's disease, Hashimoto's thyroiditis, Myasthenia gravis, Psoriasis, Lymphoproliferative disease (ALPS), Sjogren's syndrome, Autoimmune neuropathies, Gullian-Barre syndrome, Autoimmune uveitis, Autoimmune hemolytic anemia, Pernicious anemia, Aplastic anemia, Pure red cell anemia, Autoimmune thrombocytopenia, Temporal arteritis, Anti-phospholipid syndrome, Vasculitides, Wegener's granulomatosis, Behcet's disease, Dermatitis herpetiformis, Pemphigus vulgaris, Vitiligo, Primary biliary cirrhosis, Autoimmune hepatitis, Autoimmune oophoritis and orchitis, Autoimmune disease of the adrenal gland, Scleroderma, Polymyositis, Dermatomyositis, Autoimmune menagitis, Autoimmune dermatitis, Alopecia areata, Autoimmune uveitis, Allergic encephalomyelitis, Interstitial lung fibrosis, Seronegative arthropathies, Sarcoidosis, Orchitis/vasectomy reversal procedure, Raynoud's disease, Type B insulin-resistant diabetes, Antibody-mediated cytotoxicity, Type III hypersensitivity reactions, POEMS syndrome, Polyneuropathy, Organomegaly, Endocrinopathy, Monoclonal gammopathy, Skin changes syndrome, Pemphigus, Mixed connective tissue diseases, Idiopathic Addison's disease, Post-MI cardiotomy syndrome, Wilson's disease, Hemachromatosis, Alpha-1-antitrypsin deficiency, Osteoporosis, Hypothalamic-pituitary-adrenal axis evaluation, Familial hematophagocytic lymphohistiocytosis, Pre-eclampsia, OKT3 therapy, Anti-CD3 therapy, Cytokine therapy, Chemotherapy, Radiation therapy and Immune tolerance therapy via co-administration of self-antigens or self-tissues. In various embodiments, the subject is a mammal, e.g. a human.

In an aspect of this embodiment, the autoimmune disease is selected from rheumatoid arthritis, multiple sclerosis, Systemic lupus erythematosus.

In an aspect of this embodiment, the autoimmune disease is rheumatoid arthritis.

In an additional aspect of this embodiment, the subject is a mammal.

In an aspect of this embodiment, the mammal is selected from the group consisting of humans, primates, farm animals, domestic animals, and laboratory animals.

In an aspect of this embodiment, the mammal is a human.

In an additional aspect of this embodiment, the effective amount is about 0.001 mg/kg to about 50 mg/kg. Preferably, the effective amount is about 0.01 mg/kg to about 25 mg/kg. More preferably, the effective amount is about 0.1 mg/kg to about 10 mg/kg.

Another embodiment of the present invention is a method for treating autoimmune diseases in a subject. The method comprises administering to the subject an effective amount of a compound selected from the group consisting of the compounds in Table 1, and crystalline forms, hydrates, or pharmaceutically acceptable salts thereof. In various embodiments, the subject is a mammal, e.g. a human.

In one aspect of this embodiment, the autoimmune disease is selected from rheumatoid arthritis, multiple sclerosis, Systemic lupus erythematosus, Type I diabetes, Ankylosing spondylitis, Spondyloarthropathies, Crohn's disease (inflammatory bowel disease), Grave's disease, Hashimoto's thyroiditis, Myasthenia gravis, Psoriasis, Lymphoproliferative disease (ALPS), Sjogren's syndrome, Autoimmune neuropathies, Gullian-Barre syndrome, Autoimmune uveitis, Autoimmune hemolytic anemia, Pernicious anemia, Aplastic anemia, Pure red cell anemia, Autoimmune thrombocytopenia, Temporal arteritis, Anti-phospholipid syndrome, Vasculitides, Wegener's granulomatosis, Behcet's disease, Dermatitis herpetiformis, Pemphigus vulgaris, Vitiligo, Primary biliary cirrhosis, Autoimmune hepatitis, Autoimmune oophoritis and orchitis, Autoimmune disease of the adrenal gland, Scleroderma, Polymyositis, Dermatomyositis, Autoimmune menagitis, Autoimmune dermatitis, Alopecia areata, Autoimmune uveitis, Allergic encephalomyelitis, Interstitial lung fibrosis, Seronegative arthropathies, Sarcoidosis, Orchitis/vasectomy reversal procedure, Raynoud's disease, Type B insulin-resistant diabetes, Antibody-mediated cytotoxicity, Type III hypersensitivity reactions, POEMS syndrome, Polyneuropathy, Organomegaly, Endocrinopathy, Monoclonal gammopathy, Skin changes syndrome, Pemphigus, Mixed connective tissue diseases, Idiopathic Addison's disease, Post-MI cardiotomy syndrome, Wilson's disease, Hemachromatosis, Alpha-1-antitrypsin deficiency, Osteoporosis, Hypothalamic-pituitary-adrenal axis evaluation, Familial hematophagocytic lymphohistiocytosis, Pre-eclampsia, OKT3 therapy, Anti-CD3 therapy, Cytokine therapy, Chemotherapy, Radiation therapy and Immune tolerance therapy via co-administration of self-antigens or self-tissues.

In an aspect of this embodiment, the autoimmune disease is selected from rheumatoid arthritis, multiple sclerosis, Systemic lupus erythematosus.

In an aspect of this embodiment, the autoimmune disease is selected from rheumatoid arthritis.

In another aspect of this embodiment, the subject is a mammal.

In an aspect of this embodiment, the mammal is selected from the group consisting of humans, primates, farm animals, domestic animals, and laboratory animals.

In an aspect of this embodiment, the mammal is a human.

In an additional aspect of this embodiment, the effective amount is about 0.001 mg/kg to about 50 mg/kg. Preferably, the effective amount is about 0.01 mg/kg to about 25 mg/kg. More preferably, the effective amount is about 0.1 mg/kg to about 10 mg/kg.

A further embodiment of the present invention is a method for treating autoimmune diseases in a subject. The method comprises administering to the subject an effective amount of a compound selected from the group consisting of:

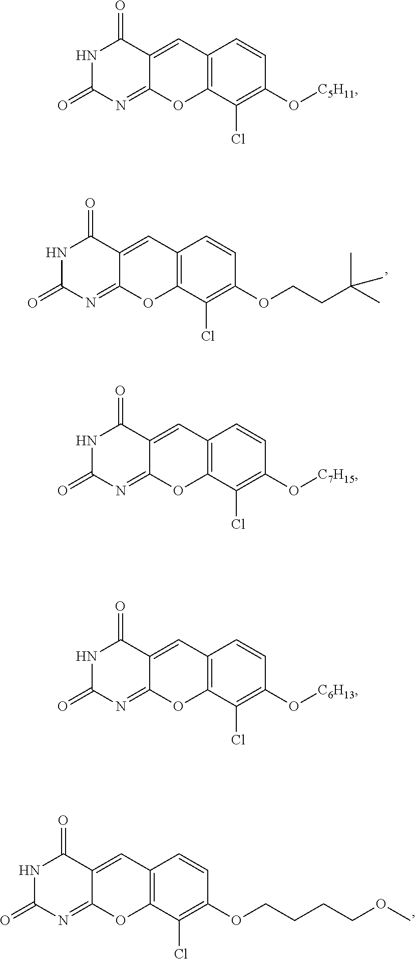

and crystalline forms, hydrates, or pharmaceutically acceptable salts thereof. In various embodiments, the subject is a mammal, e.g. a human.

In one aspect of this embodiment, the autoimmune disease is selected from rheumatoid arthritis, multiple sclerosis, Systemic lupus erythematosus, Type I diabetes, Ankylosing spondylitis, Spondyloarthropathies, Crohn's disease (inflammatory bowel disease), Grave's disease, Hashimoto's thyroiditis, Myasthenia gravis, Psoriasis, Lymphoproliferative disease (ALPS), Sjogren's syndrome, Autoimmune neuropathies, Gullian-Barre syndrome, Autoimmune uveitis, Autoimmune hemolytic anemia, Pernicious anemia, Aplastic anemia, Pure red cell anemia, Autoimmune thrombocytopenia, Temporal arteritis, Anti-phospholipid syndrome, Vasculitides, Wegener's granulomatosis, Behcet's disease, Dermatitis herpetiformis, Pemphigus vulgaris, Vitiligo, Primary biliary cirrhosis, Autoimmune hepatitis, Autoimmune oophoritis and orchitis, Autoimmune disease of the adrenal gland, Scleroderma, Polymyositis, Dermatomyositis, Autoimmune menagitis, Autoimmune dermatitis, Alopecia areata, Autoimmune uveitis, Allergic encephalomyelitis, Interstitial lung fibrosis, Seronegative arthropathies, Sarcoidosis, Orchitis/vasectomy reversal procedure, Raynoud's disease, Type B insulin-resistant diabetes, Antibody-mediated cytotoxicity, Type III hypersensitivity reactions, POEMS syndrome, Polyneuropathy, Organomegaly, Endocrinopathy, Monoclonal gammopathy, Skin changes syndrome, Pemphigus, Mixed connective tissue diseases, Idiopathic Addison's disease, Post-MI cardiotomy syndrome, Wilson's disease, Hemachromatosis, Alpha-1-antitrypsin deficiency, Osteoporosis, Hypothalamic-pituitary-adrenal axis evaluation, Familial hematophagocytic lymphohistiocytosis, Pre-eclampsia, OKT3 therapy, Anti-CD3 therapy, Cytokine therapy, Chemotherapy, Radiation therapy and Immune tolerance therapy via co-administration of self-antigens or self-tissues.

In an aspect of this embodiment, the autoimmune disease is selected from rheumatoid arthritis, multiple sclerosis, Systemic lupus erythematosus.

In an aspect of this embodiment, the autoimmune disease is rheumatoid arthritis.

In another aspect of this embodiment, the subject is a mammal.

In an aspect of this embodiment, the mammal is selected from the group consisting of humans, primates, farm animals, domestic animals, and laboratory animals.

In an aspect of this embodiment, the mammal is a human.

In an additional aspect of this embodiment, the effective amount is about 0.001 mg/kg to about 50 mg/kg. Preferably, the effective amount is about 0.01 mg/kg to about 25 mg/kg. More preferably, the effective amount is about 0.1 mg/kg to about 10 mg/kg.

An additional embodiment of the present invention is a kit for treating autoimmune diseases in a subject. The kit comprises a compound having the structure of formula (I):

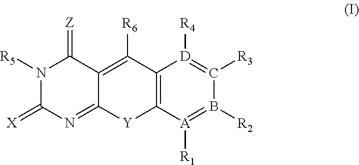

wherein:
A, B, C, and D are independently selected from the group consisting of carbon and nitrogen;
X, Y, and Z are independently selected from the group consisting of oxygen, sulfur, and NRa;
R1, R2, R3, and R4 are independently selected from the group consisting of no atom, hydrogen, halogen, C1-9 alkyl, C2-9 alkenyl, C2-9 alkynyl, aryl, heterocyclic, —OH, —ORa, —ORaORb, —ORaORbORc, —ORa(C═O)Rb—O(C═O)Ra, —O(C═O)ORa, —O(C═O)NRaRb, cyano, nitro, —CF3, —CHF2, —CH2F, —CHO, —COOH, —CORa, —COORa, —CONRaRb, —CONHCONRaRb, —NRaRb, —NHCORa, —NRbCORa, —CSOH, —CSRa, —CSORa, —CSNRaRb, —CSNHCSNRaRb, —SH, —SRa, —S(C═O)Ra, —S(C═O)ORa, —S(C═O)NRaRb;
R5 is selected from the group consisting of hydrogen, C₁₋₉ alkyl, C₂₋₉ alkenyl, C₂₋₉ alkynyl, aryl, heterocyclic, —RaCO, —RaNHCO, and —RaOCO; and
R6, Ra, Rb, and Rc are independently selected from the group consisting of hydrogen, hydroxyl, amine, C₁₋₉ alkyl, C₂₋₉ alkenyl, C₂₋₉ alkynyl, aryl, and heterocyclic, or a crystalline form, hydrate, or pharmaceutically acceptable salt thereof, packaged together with instructions for its use.

The kits may also include suitable storage containers, e.g., ampules, vials, tubes, etc., for each NF-κB inhibitor of the present invention (which may e.g., may be in the form of pharmaceutical compositions) and other reagents, e.g., buffers, balanced salt solutions, etc., for use in administering the NF-κB inhibitors to subjects. The NF-κB inhibitors of the invention and other reagents may be present in the kits in any convenient form, such as, e.g., in a solution or in a powder form. The kits may further include a packaging container, optionally having one or more partitions for housing the NF-κB inhibitors or pharmaceutical compositions of the present invention and other optional reagents.

In one aspect of this embodiment,

X, Y, and Z are independently selected from the group consisting of oxygen and sulfur;
R1 is selected from the group consisting of —H, —F, —Cl, —OMe, and —OEt;
R2 is selected from the group consisting of —H, —CH3, —OH, —OMe, —OEt, -Me, -Et, -nPr, —O-nPr, —OEtnPr, —OC4H9, —OC5H11, —OC6H13, —OC7H15, —O-isobutyl, —O— isopentyl, —OCnH2nOMe, —OCnH2nOCmH2mOMe, —OCnH2nOH, —OCnH2nOCmH2mOH, —OCnH2nOEt, —OCnH2nOCmH2mOEt, —O-CnH2nCOOH, —O-CnH2nCONH2, —O-CnH2nCONHMe,

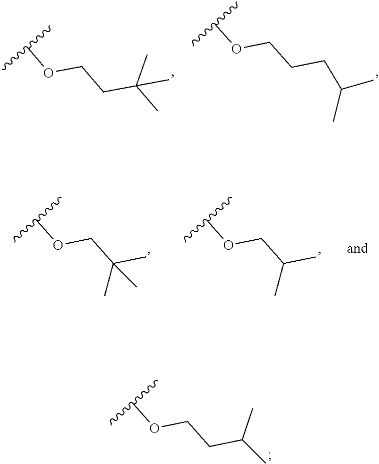

R3 is selected from the group consisting of —H, —Cl, —Br, —F, and —OMe;
R4 is selected from the group consisting of —H and —OMe;
R5 is selected from the group consisting of —H, -Me, -Et, —Pr, -iPr, -Ph, -iBu, and -nBu; and
R6 is selected from the group consisting of —H and —CH3,
m is 2, 3, 4 or 5; and,
n is 2, 3, 4, or 5.

In another aspect of this embodiment,

X, Y, and Z are independently selected from the group consisting of oxygen and sulfur; and
R₆ is hydrogen, or a crystalline form, hydrate, or pharmaceutically acceptable salt thereof.

In an aspect of this embodiment,

Z is oxygen;
R1 and R3 are selected from the group consisting of hydrogen, halogen, —CN, and —CF3;
R2 is selected from the group consisting of C₁₋₉ alkoxy, —OCnH2nOMe, —OCnH2nOCmH2mOMe, —OCnH2nOH, —OCnH2nOCmH2mOH, OCnH2nOEt, —OCnH2nOCmH2mOEt, —O-CnH2nCOOH, —O-CnH2nCONH2, —O-CnH2nCONHMe, and —OH;
m is 2, 3, 4 or 5;
n is 2, 3, 4, or 5; and
R4 is selected from the group consisting of hydrogen, C₁₋₉ alkoxy and —OH.

More preferably,

X and Y are oxygen; and
R4 is hydrogen.

In an aspect of this embodiment, the compound is selected from the group consisting of:

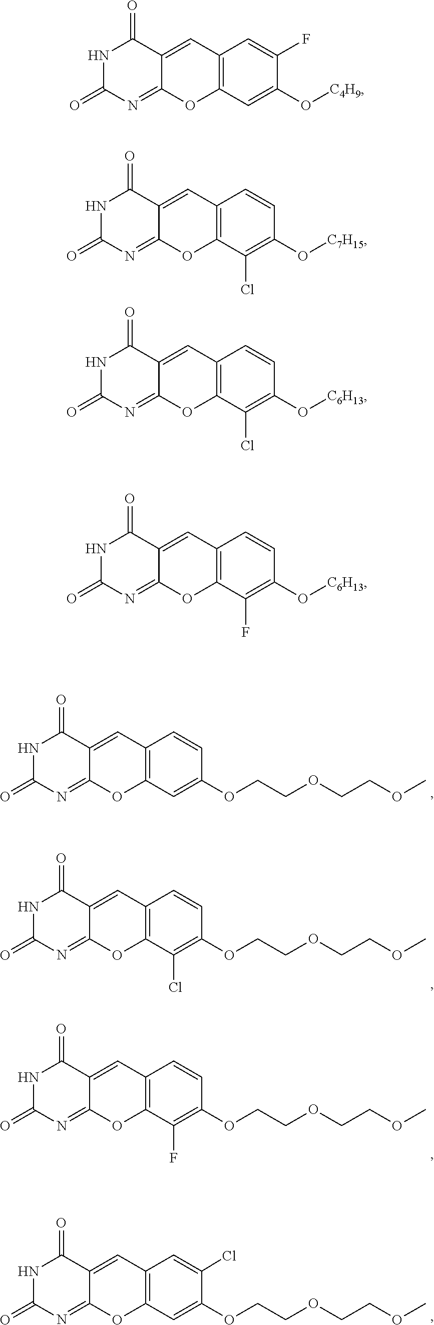

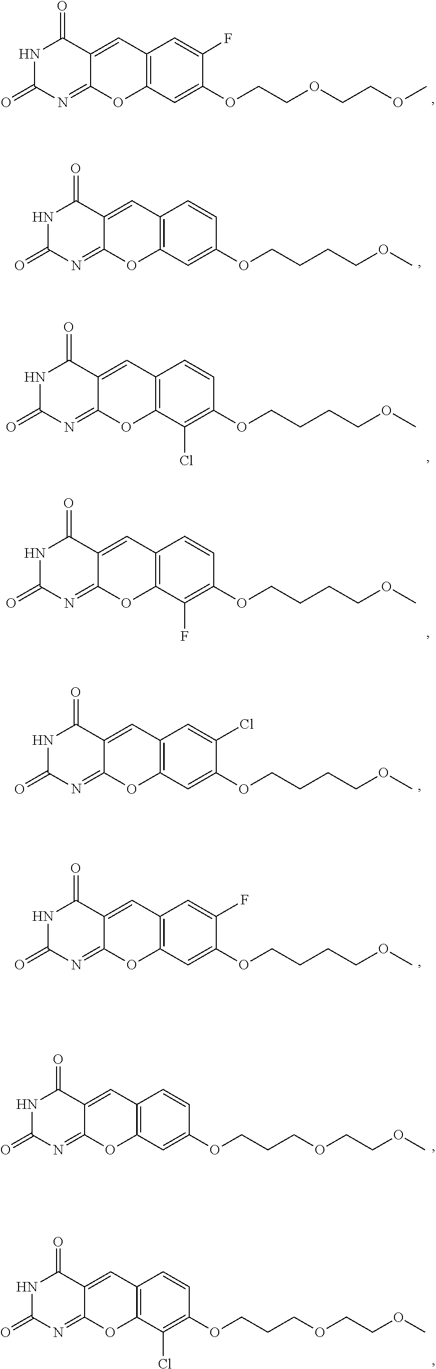

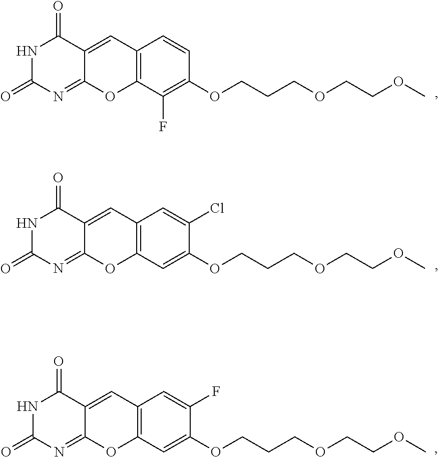 [SMILES: COCCOCCCOc1ccc2cc3c(=O)[nH]c(=O)nc-3oc2c1F ; COCCOCCCOc1cc2oc3nc(=O)[nH]c(=O)c-3cc2cc1Cl ; COCCOCCCOc1cc2oc3nc(=O)[nH]c(=O)c-3cc2cc1F]

and crystalline forms, hydrates, or pharmaceutically acceptable salts thereof.

In another aspect of this embodiment, the autoimmune disease is selected from rheumatoid arthritis, multiple sclerosis, Systemic lupus erythematosus, Type I diabetes, Ankylosing spondylitis, Spondyloarthropathies, Crohn's disease (inflammatory bowel disease), Grave's disease, Hashimoto's thyroiditis, Myasthenia gravis, Psoriasis, Lymphoproliferative disease (ALPS), Sjogren's syndrome, Autoimmune neuropathies, Gullian-Barre syndrome, Autoimmune uveitis, Autoimmune hemolytic anemia, Pernicious anemia, Aplastic anemia, Pure red cell anemia, Autoimmune thrombocytopenia, Temporal arteritis, Anti-phospholipid syndrome, Vasculitides, Wegener's granulomatosis, Behcet's disease, Dermatitis herpetiformis, Pemphigus vulgaris, Vitiligo, Primary biliary cirrhosis, Autoimmune hepatitis, Autoimmune oophoritis and orchitis, Autoimmune disease of the adrenal gland, Scleroderma, Polymyositis, Dermatomyositis, Autoimmune menagitis, Autoimmune dermatitis, Alopecia areata, Autoimmune uveitis, Allergic encephalomyelitis, Interstitial lung fibrosis, Seronegative arthropathies, Sarcoidosis, Orchitis/vasectomy reversal procedure, Raynoud's disease, Type B insulin-resistant diabetes, Antibody-mediated cytotoxicity, Type III hypersensitivity reactions, POEMS syndrome, Polyneuropathy, Organomegaly, Endocrinopathy, Monoclonal gammopathy, Skin changes syndrome, Pemphigus, Mixed connective tissue diseases, Idiopathic Addison's disease, Post-MI cardiotomy syndrome, Wilson's disease, Hemachromatosis, Alpha-1-antitrypsin deficiency, Osteoporosis, Hypothalamic-pituitary-adrenal axis evaluation, Familial hematophagocytic lymphohistiocytosis, Pre-eclampsia, OKT3 therapy, Anti-CD3 therapy, Cytokine therapy, Chemotherapy, Radiation therapy and Immune tolerance therapy via co-administration of self-antigens or self-tissues. In various embodiments, the subject is a mammal, e.g. a human.

In an aspect, the autoimmune disease is selected from rheumatoid arthritis, multiple sclerosis, Systemic lupus erythematosus.

In an aspect, the autoimmune disease is rheumatoid arthritis.

In an additional aspect of this embodiment, the subject is a mammal.

In an aspect, the mammal is selected from the group consisting of humans, primates, farm animals, domestic animals, and laboratory animals.

In an aspect, the mammal is a human.

Another embodiment of the present invention is a kit for treating autoimmune diseases in a subject. The kit comprises a compound selected from the group consisting of the compounds in Table 1, and crystalline forms, hydrates, or pharmaceutically acceptable salts thereof.

In various embodiments, the autoimmune disease is selected from rheumatoid arthritis, multiple sclerosis, Systemic lupus erythematosus, Type I diabetes, Ankylosing spondylitis, Spondyloarthropathies, Crohn's disease (inflammatory bowel disease), Grave's disease, Hashimoto's thyroiditis, Myasthenia gravis, Psoriasis, Lymphoproliferative disease (ALPS), Sjogren's syndrome, Autoimmune neuropathies, Gullian-Barre syndrome, Autoimmune uveitis, Autoimmune hemolytic anemia, Pernicious anemia, Aplastic anemia, Pure red cell anemia, Autoimmune thrombocytopenia, Temporal arteritis, Anti-phospholipid syndrome, Vasculitides, Wegener's granulomatosis, Behcet's disease, Dermatitis herpetiformis, Pemphigus vulgaris, Vitiligo, Primary biliary cirrhosis, Autoimmune hepatitis, Autoimmune oophoritis and orchitis, Autoimmune disease of the adrenal gland, Scleroderma, Polymyositis, Dermatomyositis, Autoimmune menagitis, Autoimmune dermatitis, Alopecia areata, Autoimmune uveitis, Allergic encephalomyelitis, Interstitial lung fibrosis, Seronegative arthropathies, Sarcoidosis, Orchitis/vasectomy reversal procedure, Raynoud's disease, Type B insulin-resistant diabetes, Antibody-mediated cytotoxicity, Type III hypersensitivity reactions, POEMS syndrome, Polyneuropathy, Organomegaly, Endocrinopathy, Monoclonal gammopathy, Skin changes syndrome, Pemphigus, Mixed connective tissue diseases, Idiopathic Addison's disease, Post-MI cardiotomy syndrome, Wilson's disease, Hemachromatosis, Alpha-1-antitrypsin deficiency, Osteoporosis, Hypothalamic-pituitary-adrenal axis evaluation, Familial hematophagocytic lymphohistiocytosis, Pre-eclampsia, OKT3 therapy, Anti-CD3 therapy, Cytokine therapy, Chemotherapy, Radiation therapy and Immune tolerance therapy via co-administration of self-antigens or self-tissues.

In an aspect, the autoimmune disease is selected from rheumatoid arthritis, multiple sclerosis, Systemic lupus erythematosus.

In an aspect, the autoimmune disease is rheumatoid arthritis.

In an aspect of this embodiment, the subject is a mammal.

In an aspect, the mammal is selected from the group consisting of humans, primates, farm animals, domestic animals, and laboratory animals.

In an aspect, the mammal is a human.

A further embodiment of the present invention is a kit for treating autoimmune diseases in a subject. The kit comprises a compound selected from the group consisting of:

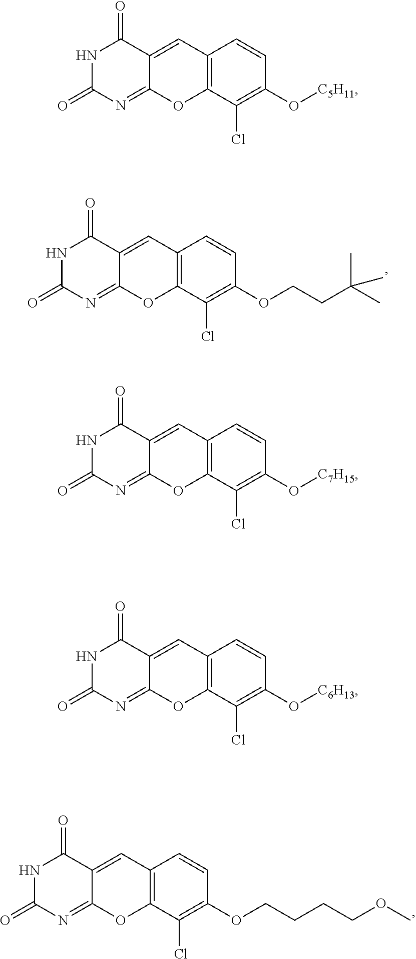

and crystalline forms, hydrates, or pharmaceutically acceptable salts thereof, packaged together with instructions for its use. In various embodiments, the subject is a mammal, e.g. a human.

In various embodiments, the autoimmune disease is selected from rheumatoid arthritis, multiple sclerosis, Systemic lupus erythematosus, Type I diabetes, Ankylosing spondylitis, Spondyloarthropathies, Crohn's disease (inflammatory bowel disease), Grave's disease, Hashimoto's thyroiditis, Myasthenia gravis, Psoriasis, Lymphoproliferative disease (ALPS), Sjogren's syndrome, Autoimmune neuropathies, Gullian-Barre syndrome, Autoimmune uveitis, Autoimmune hemolytic anemia, Pernicious anemia, Aplastic anemia, Pure red cell anemia, Autoimmune thrombocytopenia, Temporal arteritis, Anti-phospholipid syndrome, Vasculitides, Wegener's granulomatosis, Behcet's disease, Dermatitis herpetiformis, Pemphigus vulgaris, Vitiligo, Primary biliary cirrhosis, Autoimmune hepatitis, Autoimmune oophoritis and orchitis, Autoimmune disease of the adrenal gland, Scleroderma, Polymyositis, Dermatomyositis, Autoimmune menagitis, Autoimmune dermatitis, Alopecia areata, Autoimmune uveitis, Allergic encephalomyelitis, Interstitial lung fibrosis, Seronegative arthropathies, Sarcoidosis, Orchitis/vasectomy reversal procedure, Raynoud's disease, Type B insulin-resistant diabetes, Antibody-mediated cytotoxicity, Type III hypersensitivity reactions, POEMS syndrome, Polyneuropathy, Organomegaly, Endocrinopathy, Monoclonal gammopathy, Skin changes syndrome, Pemphigus, Mixed connective tissue diseases, Idiopathic Addison's disease, Post-MI cardiotomy syndrome, Wilson's disease, Hemachromatosis, Alpha-1-antitrypsin deficiency, Osteoporosis, Hypothalamic-pituitary-adrenal axis evaluation, Familial hematophagocytic lymphohistiocytosis, Pre-eclampsia, OKT3 therapy, Anti-CD3 therapy, Cytokine therapy, Chemotherapy, Radiation therapy and Immune tolerance therapy via co-administration of self-antigens or self-tissues.

In an embodiment, the autoimmune disease is selected from rheumatoid arthritis, multiple sclerosis, and systemic lupus erythematosus.

In an embodiment, the autoimmune disease is rheumatoid arthritis.

In an aspect of this embodiment, the subject is a mammal.

In an aspect of this embodiment, the mammal is selected from the group consisting of humans, primates, farm animals, domestic animals, and laboratory animals.

In an aspect of this embodiment, the mammal is a human.

### Definitions

The term “aliphatic”, as used herein, means a group composed of carbon and hydrogen atoms that does not contain aromatic rings. Accordingly, aliphatic groups include alkyl, alkenyl, alkynyl, and carbocyclyl groups.

The term “alkyl” means the radical of saturated aliphatic groups that does not have a ring structure, including straight chain alkyl groups, and branched chain alkyl groups. Alkyl groups of the present invention have at least one and up to twenty carbon atoms and can be optionally substituted with one or more heteroatoms selected from halogen, nitrogen, oxygen, and sulfur.

The term “alkyl” also refers to cyclic hydrocarbon rings having at least three, and up to twenty, carbon atoms, optionally substituted with one or more heteroatoms selected from halogen, nitrogen, oxygen, and sulfur. The cyclic hydrocarbon ring can be monocyclic, bicyclic, polycyclic or bridge cyclic. Examples of alkyl groups include, but are not limited to, methyl, ethyl, propyl, isopropyl, butyl, isobutyl, pentyl, neopentyl, cyclopropyl, cyclobutyl, 2-chlorobutyl, 3-fluoropentyl, 4-hydroxybutyl, 3-methoxypropyl, 2-methoxypropyl, 2-methylbutyl, 3-methylbutyl (isopentyl), 2-chloro-4-hydroxybutyl, 5-aminohexyl, 2,2-difluorocyclobutyl, 1,3-difluorocyclohexyl, 3-thiolhexyl, and the like.

The term “alkenyl” refers to a straight or branched hydrocarbon chain having at least three, and up to twenty, carbon atoms with at least one double bond (—C═C—), optionally substituted with one or more heteroatoms selected from halogen, nitrogen, oxygen, and sulfur.

The term “alkenyl” also refers to cyclic hydrocarbon rings having at least three, and up to twenty, carbon atoms with at least one double bond (—C═C—), optionally substituted with one or more heteroatoms selected from halogen, nitrogen, oxygen, and sulfur. Examples of alkenyl groups include, but are not limited to, ethenyl, chlorovinyl, propenyl, propenylene, allyl, 1,4-butandienyl, 1,2-cyclobutenyl, and the like.

The term “alkynyl” refers to a straight or branched hydrocarbon chain having at least three, and up to twenty, carbon atoms with at least one triple bond, with or without one or more double bond, and optionally substituted with one or more heteroatoms selected from halogen, nitrogen, oxygen, and sulfur. The term “alkenyl” also refers to cyclic hydrocarbon rings with at least one triple bond with or without one or more double bond (—C═C—) and optionally substituted with one or more heteroatoms selected from halogen, nitrogen, oxygen, and sulfur. Examples of alkynyl groups include, but are not limited to, ethynyl, propynyl, butynyl, 3-methylbutynyl, and the like.

The term “aryl” refers to monocyclic, bicyclic or polycyclic aromatic hydrocarbon ring structures optionally substituted with one or more heteroatoms selected from halogen, nitrogen, oxygen, and sulfur, and/or optionally substituted with one or more alkyl, alkenyl or alkynyl groups. Examples of aryl groups include, but are not limited to, phenyl, naphthyl, hydroxyl phenyl, chlorophenyl, 2-chloro-4-fluorophenyl, methylphenyl, cyanonaphthyl, and the like.

The term “heterocyclic” refers to saturated or unsaturated mono- or poly-carbocyclic structures in which at least one carbon atom of at least one of the rings is replaced by nitrogen, sulfur, phosphorus, or oxygen. The term “heterocyclic” is intended to encompass fully saturated and unsaturated ring systems as well as partially unsaturated ring systems, including all possible isomeric forms of the heterocycle (for example, pyrrolyl comprises 1H-pyrrolyl and 2H-pyrrolyl).

Examples of a monocyclic heterocycle (e.g., a 4-, 5-, or 6-membered ring) or a bicyclic (e.g., a 5/6, 5/5, 6/6 system) saturated heterocycle include, but are not limited to, tetrahydrofuranyl, pyrrolidinyl, tetrahydrothienyl, dihydrooxazolyl, piperidinyl, hexahydropyrimidinyl, dioxanyl, morpholinyl, dithianyl, thiomorpholinyl, piperazinyl and the like.

Examples of a partially saturated monocyclic, bicyclic or tricyclic heterocycle include, but are not limited to, pyrrolinyl, imidazolinyl, pyrazolinyl, 2,3-dihydrobenzofuranyl, 1,3-benzodioxolanyl, 2,3dihydro-1,4-benzodioxinyl, indolinyl and the like.

Examples of an aromatic monocyclic, bicyclic or tricyclic heterocycle include, but are not limited to, pyrrolyl, furanyl, thienyl, imidazolyl, oxazolyl, isoxazolyl, thiazolyl, isothiazolyl, pyrazolyl, 1,2,3-triazolyl, 1,2,5-thiadiazolyl, 1,2,3-thiadiazolyl, 1,2,3-oxadiazolyl, 1,2,5-oxadiazolyl, pyridinyl, pyrimidinyl, pyrazinyl, pyridazinyl, triazinyl, benzofuranyl, isobenzofuranyl, benzothienyl, isobenzothienyl, indolizinyl, indolyl, isoindoly, benzoxazolyl, benzimidazolyl, indazolyl, benzisoxazolyl, benzisothiazolyl, benzopyrazolyl, benzoxadiazolyl, benzothiadiazolyl, benzotriazolyl, quinolinyl, isoquinolinyl, cinnolinyl, quinolizinyl, phthalazinyl, quinoxalinyl, quinazolinyl, naphthyridinyl, pteridinyl, pyrrolopyridinyl, thienopyridinyl, furanopyridinyl, isothiazolopyridinyl, thiazolopyridinyl, isoxazolopyridinyl, oxazolopyridinyl, pyrazolopyridinyl, imidazopyridinyl, pyrrolopyrazinyl, thienopyrazinyl, furanopyrazinyl, isothiazolopyrazinyl, thiazolopyrazinyl, isoxazolopyrazinyl, oxazolopyrazinyl, pyrazolopyrazinyl, imidazopyrazinyl, pyrrolopyrimidinyl, thienopyrimidinyl, furanopyrimidinyl, isothiazolopyrimidinyl, thiazolopyrimidinyl, isoxazolopyrimidinyl, oxazolopyrimidinyl, pyrazolopyrimidinyl, imidazopyrimidinyl, pyrrolopyridazinyl, thienopyridazinyl, furanopyridazinyl, isothiazolopyridazinyl, thiazolopyridazinyl, isoxazolopyridazinyl, oxazolopyridazinyl, pyrazolopyridazinyl, imidazopyridazinyl, oxadiazolopyridinyl, thiadiazolopyridinyl, triazolopyridinyl, oxadiazolopyrazinyl, thiadiazolopyrazinyl, triazolopyrazinyl, oxadiazolopyrimidinyl, thiadiazolopyrimidinyl, triazolopyrimidinyl, oxadiazolopyridazinyl, thiadiazolopyridazinyl, triazolopyridazinyl, isoxazolotriazinyl, isothiazolotriazinyl, pyrazolotriazinyl, oxazolotriazinyl, thiazolotriazinyl, imidazotriazinyl, oxadiazolotriazinyl, thiadiazolotriazinyl, triazolotriazinyl, carbazolyl and the like.

The term “carbonyl” includes, but is not limited to, CHO (aldehyde group), COOH (carboxylic acid), COR^{a} (ketone), COOR^{a}(carboxylic ester), CONR^{a}R^{b}(amide), CONHCONR^{a}R^{b}(imide), R^{a}COX (acyl halide), and R^{a}COOCOR^{b} (acid anhydride).

The term “halogen” includes, but is not limited to, fluorine, chlorine, bromine, iodine, and astatine.

In the present invention, the term “crystalline form” means the crystal structure of a compound. A compound may exist in one or more crystalline forms, which may have different structural, physical, pharmacological, or chemical characteristics. Different crystalline forms may be obtained using variations in nucleation, growth kinetics, agglomeration, and breakage.

Nucleation results when the phase-transition energy barrier is overcome, thereby allowing a particle to form from a supersaturated solution. Crystal growth is the enlargement of crystal particles caused by deposition of the chemical compound on an existing surface of the crystal. The relative rate of nucleation and growth determine the size distribution of the crystals that are formed. The thermodynamic driving force for both nucleation and growth is supersaturation, which is defined as the deviation from thermodynamic equilibrium. Agglomeration is the formation of larger particles through two or more particles (e.g., crystals) sticking together and forming a larger crystalline structure.

The term “hydrate”, as used herein, means a solid or a semi-solid form of a chemical compound containing water in a molecular complex. The water is generally in a stoichiometric amount with respect to the chemical compound.

As used herein, “pharmaceutically acceptable salts” refer to derivatives of the compounds disclosed herein wherein the compounds are modified by making acid or base salts thereof. Examples of pharmaceutically acceptable salts include, but are not limited to, mineral or organic acid salts of basic residues such as amines; alkali or organic salts of acidic residues such as carboxylic acids; and the like. For example, such salts include salts from ammonia, L-arginine, betaine, benethamine, benzathine, calcium hydroxide, choline, deanol, diethanolamine (2,2′-iminobis(ethanol)), diethylamine, 2-(diethylamino)-ethanol, 2-aminoethanol, ethylenediamine, N-ethyl-glucamine, hydrabamine, 1H-imidazole, lysine, magnesium hydroxide, 4-(2-hydroxyethyl)-morpholine, piperazine, potassium hydroxide, 1-(2-hydroxy-ethyl)-pyrrolidine, sodium hydroxide, triethanolamine (2,2′,2″-nitrilotris(ethanol)), trometh-amine, zinc hydroxide, acetic acid, 2,2-dichloro-acetic acid, adipic acid, alginic acid, ascorbic acid, L-aspartic acid, benzenesulfonic acid, benzoic acid, 2,5-dihydroxybenzoic acid, 4-acetamido-benzoic acid, (+)-camphoric acid, (+)-camphor-10-sulfonic acid, carbonic acid, cinnamic acid, citric acid, cyclamic acid, decanoic acid, dodecylsulfuric acid, ethane-1,2-disulfonic acid, ethanesulfonic acid, 2-hydroxy-ethanesulfonic acid, ethylenediamonotetraacetic acid, formic acid, fumaric acid, galacaric acid, gentisic acid, D-glucoheptonic acid, D-gluconic acid, D-glucuronic acid, glutamic acid, glutantic acid, glutaric acid, 2-oxo-glutaric acid, glycero-phosphoric acid, glycine, glycolic acid, hexanoic acid, hippuric acid, hydrobromic acid, hydrochloric acid isobutyric acid, DL-lactic acid, lactobionic acid, lauric acid, lysine, maleic acid, (−)-L-malic acid, malonic acid, DL-mandelic acid, methanesulfonic acid, galactaric acid, naphthalene-1,5-disulfonic acid, naphthalene-2-sulfonic acid, 1-hydroxy-2-naphthoic acid, nicotinic acid, nitric acid, octanoic acid, oleic acid, orotic acid, oxalic acid, palmitic acid, pamoic acid (embonic acid), phosphoric acid, propionic acid, (−)-L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid and undecylenic acid. Further pharmaceutically acceptable salts can be formed with cations from metals like aluminum, calcium, lithium, magnesium, potassium, sodium, zinc and the like. (Pharmaceutical salts, Berge, S. M. et al., J. Pharm. Sci., (1977), 66, 1-19).

The pharmaceutically acceptable salts of the present invention can be synthesized from a compound disclosed herein which contains a basic or acidic moiety by conventional chemical methods. Generally, such salts can be prepared by reacting the free acid or base forms of these compounds with a sufficient amount of the appropriate base or acid in water or in an organic diluent like ether, ethyl acetate, ethanol, isopropanol, or acetonitrile, or a mixture thereof.

The compositions of the invention comprise one or more active ingredients in admixture with one or more pharmaceutically acceptable diluents or carriers and, optionally, one or more other compounds, drugs, ingredients and/or materials. Regardless of the route of administration selected, the agents/compounds of the present invention are formulated into pharmaceutically-acceptable dosage forms by conventional methods known to those of skill in the art. See, e.g., Remington, The Science and Practice of Pharmacy (21st Edition, Lippincott Williams and Wilkins, Philadelphia, Pa.).

Pharmaceutically acceptable diluents or carriers are well known in the art (see, e.g., Remington, The Science and Practice of Pharmacy (21st Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable diluent or carrier used in a pharmaceutical composition of the invention must be “acceptable” in the sense of being compatible with the other ingredients of the formulation and not injurious to the subject. Diluents or carriers suitable for a selected dosage form and intended route of administration are well known in the art, and acceptable diluents or carriers for a chosen dosage form and method of administration can be determined using ordinary skill in the art.

The compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

The compositions of the present invention suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable diluents or carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

The compositions of the present invention for rectal or vaginal administration may be presented as a suppository, which may be prepared by mixing one or more active ingredient(s) with one or more suitable nonirritating diluents or carriers which are solid at room temperature, but liquid at body temperature and, therefore, will melt in the rectum or vaginal cavity and release the active compound. The pharmaceutical compositions of the present invention which are suitable for vaginal administration also include pessaries, tampons, creams, gels, pastes, foams or spray formulations containing such pharmaceutically-acceptable diluents or carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable diluent or carrier. The ointments, pastes, creams and gels may contain excipients. Powders and sprays may contain excipients and propellants.

The compositions of the present invention suitable for parenteral administrations may comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These pharmaceutical compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

Any formulation of the invention may be presented in unit-dose or multi-dose sealed containers, for example, ampules and vials, and may be stored in a lyophilized condition requiring only the addition of the sterile liquid diluent or carrier, for example water for injection, immediately prior to use. Extemporaneous injection solutions and suspensions may be prepared from sterile powders, granules and tablets of the type described above.

The methods of the present invention may further comprise administering to the subject at least one additional agent. Likewise, the kits of the present invention may further comprise at least one additional agent. In the methods and kits of the present invention, the additional agent may be selected from the group consisting of an antibody or fragment thereof, a cytotoxic agent, including, but not limited to, chemotherapeutic agents, a targeted agent, including, but not limited to, targeted therapeutic agents, a toxin, a radionuclide, an immunomodulator, a radiosensitizing agent, a hormone, an anti-angiogenesis agent, and combinations thereof.

As used herein, an “antibody” encompasses naturally occurring immunoglobulins as well as non-naturally occurring immunoglobulins, including, for example, single chain antibodies, chimeric antibodies (e.g., humanized murine antibodies), and heteroconjugate antibodies (e.g., bispecific antibodies). Fragments of antibodies include those that bind antigen, (e.g., Fab′, F(ab′)2, Fab, Fv, and rIgG). See also, e.g., Pierce Catalog and Handbook, 1994-1995 (Pierce Chemical Co., Rockford, Ill.); Kuby, J., Immunology, 3rd Ed., W.H. Freeman & Co., New York (1998). The term antibody also includes bivalent or bispecific molecules, diabodies, triabodies, and tetrabodies. The term “antibody” further includes both polyclonal and monoclonal antibodies.

Non-limiting examples of antibodies that may be used in the present invention include rituximab (e.g. marketed as Rituxan), cetuximab (e.g. marketed as Erbitux), bevacizumab (e.g. marketed as Avastin), necitumumab, nivolumab, pembrolizumab, and atezolizumab.

Cytotoxic agents according to the present invention include, but are not limited to, DNA damaging agents, antimetabolites, anti-microtubule agents, and antibiotic agents.

DNA damaging agents include alkylating agents, platinum-based agents, intercalating agents, and inhibitors of DNA replication. Non-limiting examples of DNA alkylating agents include cyclophosphamide, mechlorethamine, uramustine, melphalan, chlorambucil, ifosfamide, carmustine, lomustine, streptozocin, busulfan, temozolomide, pharmaceutically acceptable salts thereof, prodrugs, and combinations thereof. Non-limiting examples of platinum-based agents include cisplatin, carboplatin, oxaliplatin, nedaplatin, satraplatin, triplatin tetranitrate, pharmaceutically acceptable salts thereof, prodrugs, and combinations thereof. Non-limiting examples of intercalating agents include doxorubicin, daunorubicin, idarubicin, mitoxantrone, pharmaceutically acceptable salts thereof, prodrugs, and combinations thereof. Non-limiting examples of inhibitors of DNA replication include irinotecan, topotecan, amsacrine, etoposide, etoposide phosphate, teniposide, pharmaceutically acceptable salts thereof, prodrugs, and combinations thereof.

Antimetabolites include folate antagonists such as methotrexate and premetrexed, purine antagonists such as 6-mercaptopurine, dacarbazine, and fludarabine, and pyrimidine antagonists such as 5-fluorouracil, arabinosylcytosine, capecitabine, gemcitabine, decitabine, pharmaceutically acceptable salts thereof, prodrugs, and combinations thereof.

Anti-microtubule agents include without limitation vinca alkaloids, paclitaxel (e.g. marketed as Taxol®), docetaxel (e.g. marketed as Taxotere®), and ixabepilone (e.g. marketed as Ixempra®).

Antibiotic agents include without limitation include actinomycin, anthracyclines, valrubicin, epirubicin, bleomycin, plicamycin, mitomycin, pharmaceutically acceptable salts thereof, prodrugs, and combinations thereof.

Targeted agents of the present invention include, but are not limited to, EGFR inhibitors, such as gefitinib, erlotinib, afatinib, and osimetinib, as well as ALK inhibitors, such as crizotinib, ceritinib, and alectinib. Targeted agents of the present invention also include, for example, sorafenib.

In the present invention, the term “toxin” means an antigenic poison or venom of plant or animal origin. An example is diphtheria toxin or portions thereof.

In the present invention, the term “radionuclide” means a radioactive substance administered to the subject, e.g., intravenously or orally, after which it penetrates via the subject's normal metabolism into the target organ or tissue, where it delivers local radiation for a short time. Examples of radionuclides include, but are not limited to, I-125, At-211, Lu-177, Cu-67, I-131, Sm-153, Re-186, P-32, Re-188, In-114m, and Y-90.

In the present invention, the term “immunomodulator” means a substance that alters the immune response by augmenting or reducing the ability of the immune system to produce antibodies or sensitized cells that recognize and react with the antigen that initiated their production. Immunomodulators may be recombinant, synthetic, or natural preparations and include cytokines, corticosteroids, cytotoxic agents, thymosin, and immunoglobulins. Some immunomodulators are naturally present in the body, and certain of these are available in pharmacologic preparations. Examples of immunomodulators include, but are not limited to, granulocyte colony-stimulating factor (G-CSF), interferons, imiquimod and cellular membrane fractions from bacteria, IL-2, IL-7, IL-12, CCL3, CCL26, CXCL7, and synthetic cytosine phosphate-guanosine (CpG). Additional immunomodulators of the present invention include, but are not limited to, immune checkpoint inhibitors. Immune checkpoint inhibitors of the present invention include, but are not limited to, antibodies against, and antagonists of, CTLA-4, PD-1, PD-L1, LAG3, IDO1, and the like.

In the present invention, the term “radiosensitizing agent” means a compound that makes tumor cells more sensitive to radiation therapy. Examples of radiosensitizing agents include misonidazole, metronidazole, tirapazamine, and trans sodium crocetinate.

In the present invention, the term “hormone” means a substance released by cells in one part of a body that affects cells in another part of the body. Examples of hormones include, but are not limited to, prostaglandins, leukotrienes, prostacyclin, thromboxane, amylin, antimullerian hormone, adiponectin, adrenocorticotropic hormone, angiotensinogen, angiotensin, vasopressin, atriopeptin, brain natriuretic peptide, calcitonin, cholecystokinin, corticotropin-releasing hormone, encephalin, endothelin, erythropoietin, follicle-stimulating hormone, galanin, gastrin, ghrelin, glucagon, gonadotropin-releasing hormone, growth hormone-releasing hormone, human chorionic gonadotropin, human placental lactogen, growth hormone, inhibin, insulin, somatomedin, leptin, liptropin, luteinizing hormone, melanocyte stimulating hormone, motilin, orexin, oxytocin, pancreatic polypeptide, parathyroid hormone, prolactin, prolactin releasing hormone, relaxin, renin, secretin, somatostain, thrombopoietin, thyroid-stimulating hormone, testosterone, dehydroepiandrosterone, androstenedione, dihydrotestosterone, aldosterone, estradiol, estrone, estriol, cortisol, progesterone, calcitriol, and calcidiol.

Some compounds interfere with the activity of certain hormones or stop the production of certain hormones. These hormone-interfering compounds include, but are not limited to, tamoxifen (Nolvadex®), anastrozole (Arimidex®), letrozole (Femara®), and fulvestrant (Faslodex®). Such compounds are also within the meaning of hormone in the present invention.

As used herein, an anti-angiogenesis agent means a substance that reduces or inhibits the growth of new blood vessels, such as, e.g., an inhibitor of vascular endothelial growth factor (VEGF) and an inhibitor of endothelial cell migration. Anti-angiogenesis agents include without limitation 2-methoxyestradiol, angiostatin, bevacizumab, cartilage-derived angiogenesis inhibitory factor, endostatin, IFN-α, IL-12, itraconazole, linomide, platelet factor-4, prolactin, SU5416, suramin, tasquinimod, tecogalan, tetrathiomolybdate, thalidomide, thrombospondin, thrombospondin, TNP-470, ziv-aflibercept, pharmaceutically acceptable salts thereof, prodrugs, and combinations thereof.

The following examples are provided to further illustrate the methods of the present invention. These examples are illustrative only and are not intended to limit the scope of the invention in any way.

### EXAMPLES

### Example 1: Evaluation of IT-848 Efficacy in Collagen-Induced Arthritis (CIA) in DBA/1 Mice

The study was conducted in Collagen-Induced Arthritis (CIA) model in DBA/1 mice. The collagen-induced arthritis (CIA) mouse model is the most commonly studied autoimmune model of rheumatoid arthritis. DBA/1 mouse model was established as in the protocol described by D. D. Brand et al. Nature Protocol, 2007, 2(5), 1269.

The study compared the efficacy of IT-848 with JAK inhibitor drug, Tofacitnib (Tofa) and glucocorticoid, Dexamethasone (Dex).

A: Animal Model Preparation and Disease Induction

1: Animal Adaptation

After arriving at the lab facility, all animals were labeled with an ear tag. After 1 week's acclimation, except for normal group, the CIA mice were immunized twice, with a 21 day Interval.

2: Reagent Preparation (For Model Induction)

Acetic acid preparation: 2 mol acetic acid stock solution was diluted to a 100 mM acetic acid working solution, 0.22-micron filter sterilized and stored at 4° C. Bovine Collagen type II (CII) solution: CII was dissolved in 100 mM acetic acid to final concentration of 8 mg/mL overnight at 4° C.CII/CFA (Complete Freund's Adjuvant). Emulsion preparation: A high-speed homogenizer was used to emulsify CII solution with an equal volume of 1 mg/mL CFA on ice at 30000 r/min until stable (about 60 minutes).

3: Model Set Up

The first immunization: Mice were anesthetized with isoflurane and immunized with 50 μL of 4 mg/mL bovine CII/CFA emulsion (containing 200 μg bovine CII) subcutaneously on the base of the tail. The second immunization: Mice were again anesthetized with isoflurane and immunized a second time with 50 μL of 4 mg/mL CII/CFA emulsion, adjacent to the first injection site on the base of the tail. Normal group animals were not injected.

B: Compounds Preparation

1. Vehicle Preparation

1.1. Vehicle (a) for IT-848 (10% DMA/40% PEG400/50% H₂O)

10.0 mL DMA was mixed with 40.0 mL PEG400, then vortexed and sonicated. 50.0 mL H₂O were added to make the final volume of 100.0 mL. An appropriate volume of IN Na2CO3 was added to make the pH 8. The vehicle solution was stored at 4° C.

1.2 Vehicle (b) for Tofa and Dex (0.5% MC/0.025% Tween20)

0.5 g Methylcellulose (MC) was weighed out and added into 70.0 mL sterile water and mixed gently until it dissolved. Then, 0.025 mL Tween20 was added and water was added to make the final volume 100.0 mL. The vehicle solution was stored at 4° C.

2. Compounds (Drugs) Preparation

2.1. IT-848 (0.5 mg/mL): For Group 5, 5 mg/kg BID regimen

7.5 mg IT-848 was weighed out and added to 1.5 mL DMA, the mixture was vortexed and sonicated for 5 mins, 6.0 mL PEG400 was added, the mixture was vortexed and sonicated for 5 mins, 0.0215 mL IN Na2CO3 was added, the mixture was vortexed and sonicated until clear, 7.4785 mL sterile water was added to make the final volume 15.0 mL. The mixture was vortexed and sonicated to clear; the pH was 8. The solution was prepared freshly before dosing and protected from light.

2.2. IT-848 (0.25 mg/mL): For Group 4, 2.5 mg/kg BID regimen

6.0 mL of IT-848 (0.5 mg/mL) and 6.0 mL of vehicle (a) were added to the glass vial. The mixture was stirred or shaken gently to mix. The solution was diluted freshly before dosing and protected from light.

2.3. IT-848 (0.125 mg/mL): For Group 3, 1.25 mg/kg BID regimen

4.0 mL of IT-848 (0.25 mg/mL) and 4.0 mL of vehicle (a) were added to the glass vial. The mixture was stirred or shaken gently to mix. The solution was diluted freshly before dosing and protected from light.

2.4. Tofacitinib (3.0 mg/mL)

24.0 mg tofacitinib was weighed out and added into 8.0 mL vehicle (b), and the mixture was vortexed and sonicated for a few minutes until it dissolved. The volume was made to a final volume of 8.0 mL. The solution was prepared freshly before dosing (twice daily for BID regimen) and protected from light.

2.5. Dexamethasone (0.02 mg/mL)

0.75 mg dexamethasone was added into 37.5 mL vehicle (b), vortexed and sonicated for a few minutes until it dissolved. The final concentration was made 0.02 mg/mL, and the mixture was stored at 4° C. before use.

3. Treatments

60 immunized animals were randomized into 6 groups (N=10) upon meeting the disease criteria (1≤clinical score ≤2) following the second immunization. The day of grouping and starting dosing was the same day, and we set the day as Day 0. There were 4 or 5 batches of grouping in total. The last day of dosing was Day 13. All the compounds-treatment group including vehicle treatments were delivered with oral gavage. The dosage and dosing routes we used are indicated in Table A.

TABLE A
Grouping and dosage regimen

| | | | Dosing | Dosing | | Dosing |
|---|---|---|---|---|---|---|
| | Compound | Animal | Volume | amount | Dosing | regimen & |
| Group | Treatment | number | mL/kg | mg/kg | route | Vehicle |
| 1 | Normal | 10 | — | — | — | — |
| 2 | Vehicle | 10 | 10 | — | P.O. | BID, |
| | | | | | | 14 days |
| 3 | IT-848 | 10 | 10 | 1.25 | P.O. | BID, |
| | | | | | | 14 days |
| 4 | IT-848 | 10 | 10 | 2.5 | P.O. | BID, |
| | | | | | | 14 days |
| 5 | IT-848 | 10 | 10 | 5 | P.O. | BID, |
| | | | | | | 14 days |
| 6 | Tofacitinib | 10 | 10 | 30 | P.O. | BID, |
| | | | | | | 14 days |
| 7 | Dexa- | 10 | 10 | 0.2 | P.O. | QD, |
| | methasone | | | | | 14 days |

C: Record and Analysis

1. Body Weights

All animals were weighed before dosing daily, beginning with Day 0 to Day 14. The first day of dosing was considered Day 0, and the day after the final dosing was Day 14.

2. Clinical Score

After disease onset, the disease was assessed by a grading system (Table 2) according to severity on the paws every day.

TABLE 2
Scoring system for evaluation of arthritis severity in CIA
model

| Score | Clinical signs |
|---|---|
| 0 | No evidence of erythema and swelling |
| 1 | Erythema and mild swelling confined to the mid-foot |
| | (tarsal) or ankle joints or limited to individual digits. |
| 2 | Erythema and mild swelling extending from the ankle to |
| | the mid-foot, or at least 2 digits. |
| 3 | Erythema and moderate swelling extending from ankle to |
| | the metatarsal joints. |
| 4 | Erythema and severe swelling encompass the ankle, foot, |
| | and digits. |

3. Foot Pad Swelling Measurement

The left and right hind paw volume were measured on days 0, 3, 6, 9, 12 and day 14. The average number of left and right paw volume were calculated. All the data were analyzed and plotted into prism files.

4. Blood Collection and ELISA Assay

Serum was collected for cytokines analysis according to the following schedule. Time point 1: Serum cytokine study by ELISA (IL-6, TNF-α), 5 mice per group were randomly selected, 35 samples in total; on the day of grouping. Time point 2: Serum cytokine study by ELISA (IL-6, TNF-α), 5 mice per group were randomly selected, 35 samples in total; on day 7 of the treatment at 2 hours post 1st dose of the day. Time point 3: Serum cytokine study by ELISA (IL-6, TNF-α), the other 5 mice per group were selected that were not selected for time point 2, 35 samples in total; on day 7 of the treatment, at 8 hours post 1st dose of the day. Time point 4: On day 14 of the treatment 24 h after the last dosing, all the mice were sacrificed by CO₂. Blood were collected by heart puncture, and serum was then isolated for cytokine (IL-6, TNF-α) analysis by ELISA.

5. Histopathology of Paws

70 Mice hind paws were collected and fixed in 10% neutral buffered formalin for 24 hours followed by decalcification with 10% formic acid. Mice joint tissues were then dehydrated through a series of ascending ethanol solutions and embedded in paraffin. Sections were obtained using a rotary microtome and stained with Hematoxylin and Eosin (HE) solution for histopathology. 140 hind paws for HE staining were sectioned and subjected to pathology analysis.

D. RESULTS: Results of this experiment are shown in the figures

### DOCUMENTS

1. Aly, H. M. and Kamal, M. M. (2012). Efficient one-pot preparation of novel fused chromeno[2,3-d]pyrimidine and pyrano[2,3-d]pyrimidine derivatives. European Journal of Medicinal Chemistry 47: 18-23.
2. I. Campbell, et al. J Clin Invest. 2000, 105(12), 1799.
3. S. Ouk et al. Future Med. Chem. 2009, 1(9), 1683
4. K. Umezawa, et al. Biomed. Pharmacother. 2011, 65, 252
5. International Multiple Sclerosis Genetics Consortium, Am. J. Hum. Genet. 2013, 92, 854-865
6. G. Ellrichman et al. J. Neuroinflammation 2012, 9:15
7. N. Sanadgol et al. Toxico. Appl. Pharmacol. 2018, 842, 86
8. H. D. Brightbill, et. al. Nature Comm. 2018; 19:179]
9. R. K. Mishra Nephrol Open J. 2016; 2(1): 9-13
10. J. L. Low et al. Immunology and Cell Biology 2016, 94, 66-78

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

What is claimed is:

1. A method for treating autoimmune diseases in a subject, comprising administering to the subject an effective amount of a compound having the structure of formula (I): 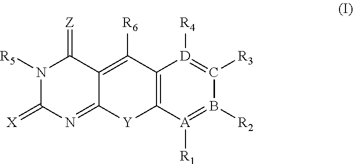
wherein:
A, B, C, and D are independently selected from the group consisting of carbon and nitrogen;
X, Y, and Z are independently selected from the group consisting of oxygen, sulfur, and NR^{a};
R₁, R₂, R₃, and R₄ are independently selected from the group consisting of no atom, hydrogen, halogen, C₁₋₉ alkyl, C₂₋₉ alkenyl, C₂₋₉ alkynyl, aryl, heterocyclic, —OH, —OR^{a}, —OR^{a}OR^{b}, —OR^{a}OR^{b}OR^{c}, —OR^{a}(C═O)R^{b}—O(C═O)R^{a}, —O(C═O)OR^{a}, —O(C═O)NR^{a}R^{b}, cyano, nitro, —CF₃, —CHF₂, —CH₂F, —CHO, —COOH, —COR^{a}, —COOR^{a}, —CONR^{a}R^{b}, —CONHCONR^{a}R^{b}, —NR^{a}R^{b}, —NHCOR^{a}, —NR^{b}COR^{a}, —CSOH, —CSR^{a}, —CSOR^{a}, —CSNR^{a}R^{b}, —CSNHCSNR^{a}R^{b}, —SH, —SR^{a}, —S(C═O)R^{a}, —S(C═O)OR^{a}, and —S(C═O)NR^{a}R^{b};
R₅ is selected from the group consisting of hydrogen, C₁₋₉ alkyl, C₂₋₉ alkenyl, C₂₋₉ alkynyl, aryl, heterocyclic, —R^{a}CO, —R^{a}NHCO, and —R^{a}OCO; and
R₆, R^{a}, R^{b}, and R^{c} are independently selected from the group consisting of hydrogen, hydroxyl, amine, C₁₋₉ alkyl, C₂₋₉ alkenyl, C₂₋₉ alkynyl, aryl, and heterocyclic,
or a crystalline form, hydrate, or pharmaceutically acceptable salt thereof.

2. The method according to claim 1,
wherein:
X, Y, and Z are independently selected from the group consisting of oxygen and sulfur;
R₁ is selected from the group consisting of —H, —F, —Cl, —OMe, and —OEt;
R₂ is selected from the group consisting of —H, —CH₃, —OH, —OMe, —OEt, -Me, -Et, -nPr, —O-nPr, —OEtnPr, —OC₄H₉, —OC₅H₁₁, —OC₆H₁₃, —OC₇H₁₅, —O-isobutyl, —O-isopentyl, —OCₙH₂ₙOMe, —OCₙH₂ₙOCₘH₂ₘOMe, —OCₙH₂ₙOH, —OCₙH₂ₙOCₘH₂ₘOH, —OCₙH₂ₙOEt, —OCₙH₂ₙOCₘH₂ₘOEt, —O—CₙH₂ₙCOOH, —O—CₙH₂ₙCONH₂, —O—CₙH₂ₙCONHMe, 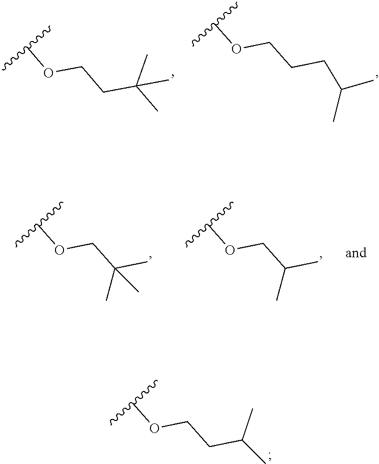
R₃ is selected from the group consisting of —H, —Cl, —Br, —F, and —OMe;
R₄ is selected from the group consisting of —H and —OMe;
R₅ is selected from the group consisting of —H, -Me, -Et, —Pr, -iPr, -Ph, -iBu, and -nBu; and
R₆ is selected from the group consisting of —H and —CH₃,
m is 2, 3, 4 or 5;
n is 2, 3, 4, or 5;
or a crystalline form, hydrate, or pharmaceutically acceptable salt thereof.

3. The method according to claim 1,
wherein:
X, Y, and Z are independently selected from the group consisting of oxygen and sulfur; and
R₆ is hydrogen,
or a crystalline form, hydrate, or pharmaceutically acceptable salt thereof.

4. The method according to claim 3,
wherein:
Z is oxygen;
R₁ and R₃ are selected from the group consisting of hydrogen, halogen, —CN, and —CF₃;
R₂ is selected from the group consisting of C₁₋₉ alkoxy, —OCₙH₂ₙOMe, —OCₙH₂ₙOCₘH₂ₘOMe, —OCₙH₂ₙOH, —OCₙH₂ₙOCₘH₂ₘOH, OCₙH₂ₙOEt, —OCₙH₂ₙOCₘH₂ₘOEt, —O—CₙH₂ₙCOOH, —O—CₙH₂ₙCONH₂, —O—CₙH₂ₙCONHMe, and —OH;
m is 2, 3, 4 or 5;
n is 2, 3, 4, or 5; and
R₄ is selected from the group consisting of hydrogen, C₁₋₉ alkoxy and —OH,
or a crystalline form, hydrate, or pharmaceutically acceptable salt thereof.

5. The method according to claim 4,
wherein:
X and Y are oxygen; and
R₄ is hydrogen,
or a crystalline form, hydrate, or pharmaceutically acceptable salt thereof.

6. The method according to claim 5, wherein the compound is selected from the group consisting of: 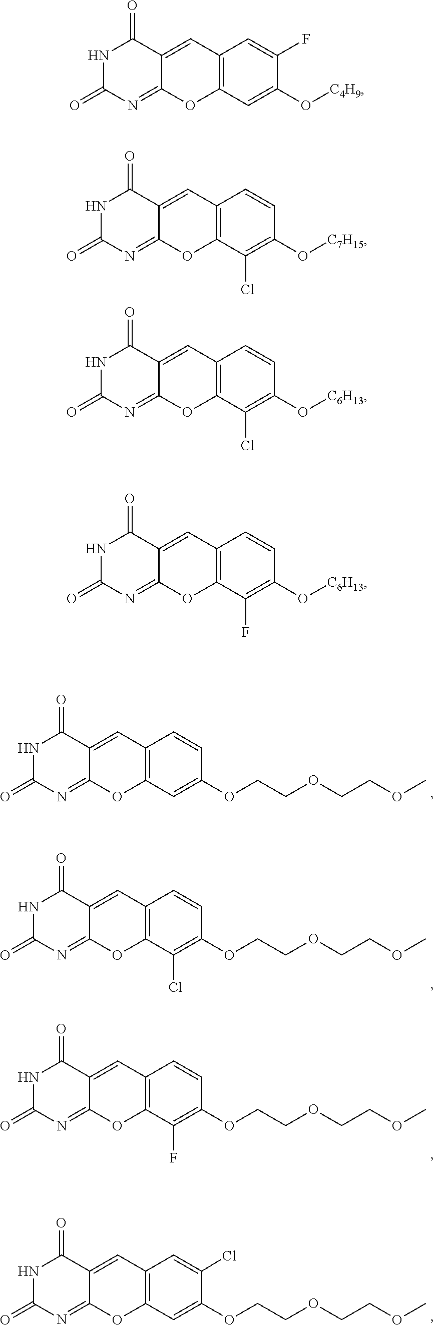 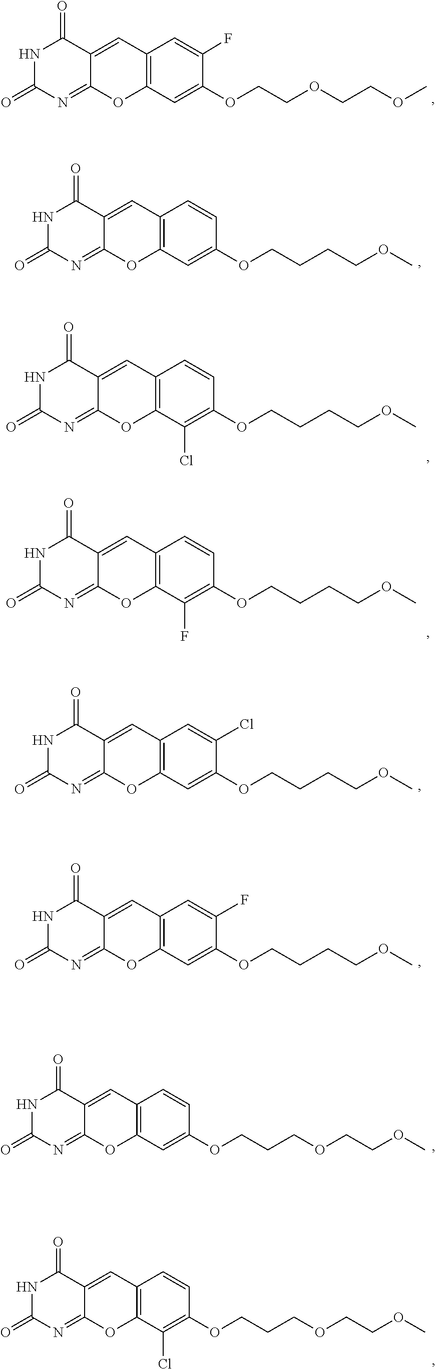 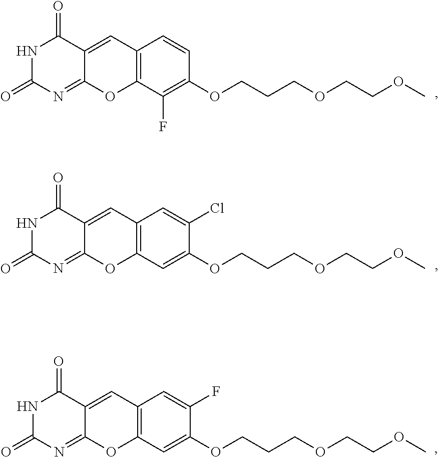 [SMILES: COCCOCCCOc1ccc2cc3c(=O)[nH]c(=O)nc-3oc2c1F ; COCCOCCCOc1cc2oc3nc(=O)[nH]c(=O)c-3cc2cc1Cl ; COCCOCCCOc1cc2oc3nc(=O)[nH]c(=O)c-3cc2cc1F]
and crystalline forms, hydrates, or pharmaceutically acceptable salts thereof.

7. The method according to claim 1, wherein the autoimmune disease is selected from the group consisting of rheumatoid arthritis, multiple sclerosis, systemic lupus erythematosus, Type I diabetes, Ankylosing spondylitis, Spondyloarthropathies, Crohn's disease (inflammatory bowel disease), Grave's disease, Hashimoto's thyroiditis, Myasthenia gravis, Psoriasis, Lymphoproliferative disease (ALPS), Sjogren's syndrome, Autoimmune neuropathies, Gullian-Barre syndrome, Autoimmune uveitis, Autoimmune hemolytic anemia, Pernicious anemia, Aplastic anemia, Pure red cell anemia, Autoimmune thrombocytopenia, Temporal arteritis, Anti-phospholipid syndrome, Vasculitides, Wegener's granulomatosis, Behcet's disease, Dermatitis herpetiformis, Pemphigus vulgaris, Vitiligo, Primary biliary cirrhosis, Autoimmune hepatitis, Autoimmune oophoritis and orchitis, Autoimmune disease of the adrenal gland, Scleroderma, Polymyositis, Dermatomyositis, Autoimmune menagitis, Autoimmune dermatitis, Alopecia areata, Autoimmune uveitis, Allergic encephalomyelitis, Interstitial lung fibrosis, Seronegative arthropathies, Sarcoidosis, Orchitis/vasectomy reversal procedure, Raynoud's disease, Type B insulin-resistant diabetes, Antibody-mediated cytotoxicity, Type III hypersensitivity reactions, POEMS syndrome, Polyneuropathy, Organomegaly, Endocrinopathy, Monoclonal gammopathy, Skin changes syndrome, Pemphigus, Mixed connective tissue diseases, Idiopathic Addison's disease, Post-MI cardiotomy syndrome, Wilson's disease, Hemachromatosis, Alpha-1-antitrypsin deficiency, Osteoporosis, Hypothalamic-pituitary-adrenal axis evaluation, Familial hematophagocytic lymphohistiocytosis, Pre-eclampsia, OKT3 therapy, Anti-CD3 therapy, Cytokine therapy, Chemotherapy, Radiation therapy and Immune tolerance therapy via co-administration of self-antigens or self-tissues.

8. The method according to claim 7, wherein the autoimmune disease is rheumatoid arthritis.

9. The method according to claim 7, wherein the autoimmune disease is multiple sclerosis.

10. The method according to claim 7, wherein the autoimmune disease is systemic lupus erythematosus.

11. The method according to claim 1, wherein the subject is a mammal.

12. The method according to claim 11, wherein the mammal is selected from the group consisting of humans, primates, farm animals, domestic animals, and laboratory animals.

13. The method according to claim 12, wherein the mammal is a human.

14. The method according to claim 1, wherein the effective amount is about 0.001 mg/kg to about 50 mg/kg.

15. A method for treating autoimmune disease in a subject, comprising administering to the subject an effective amount of a compound selected from the group consisting of: 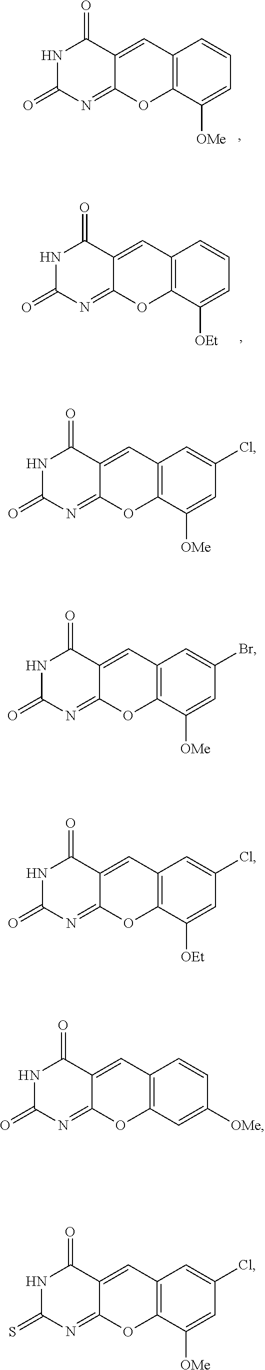 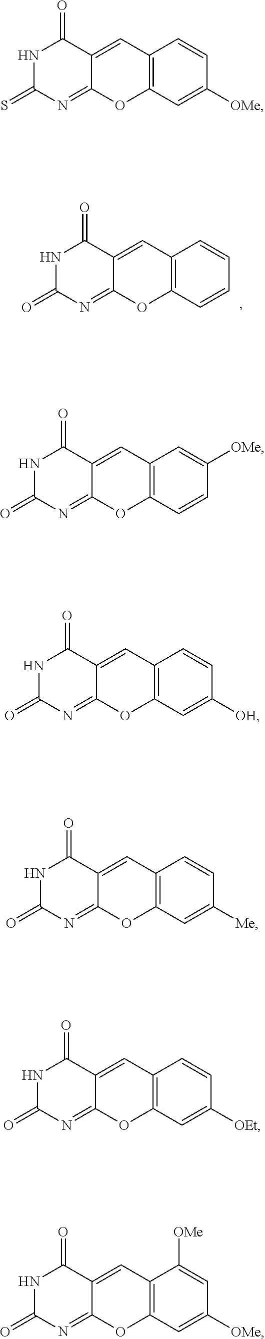 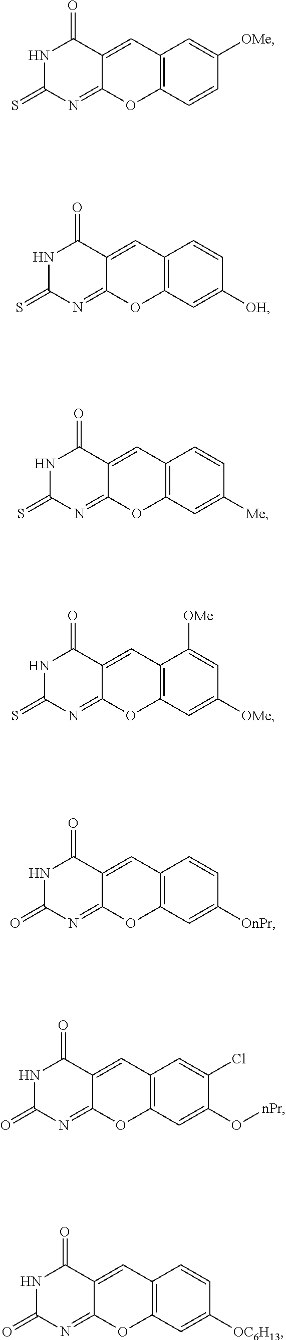 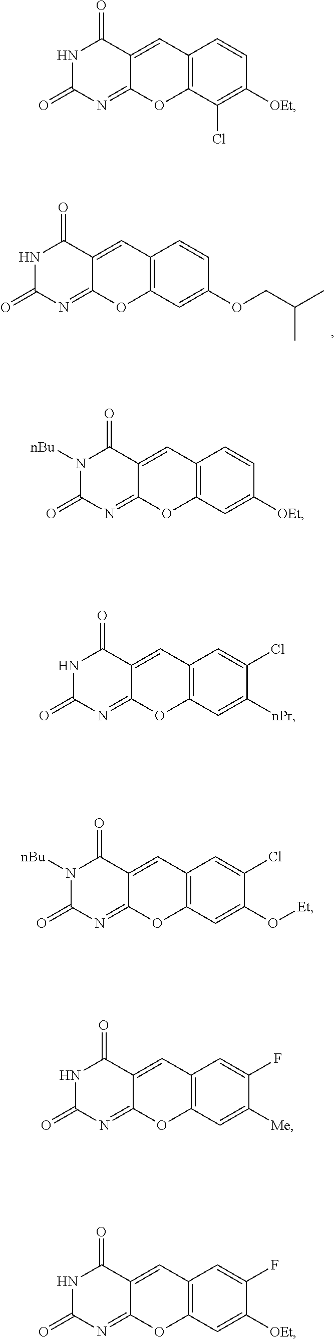 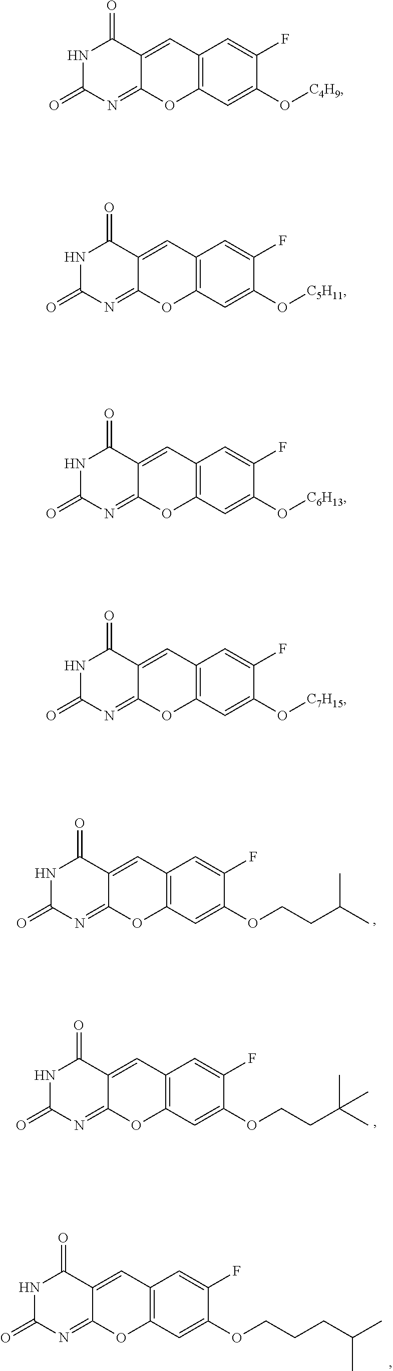 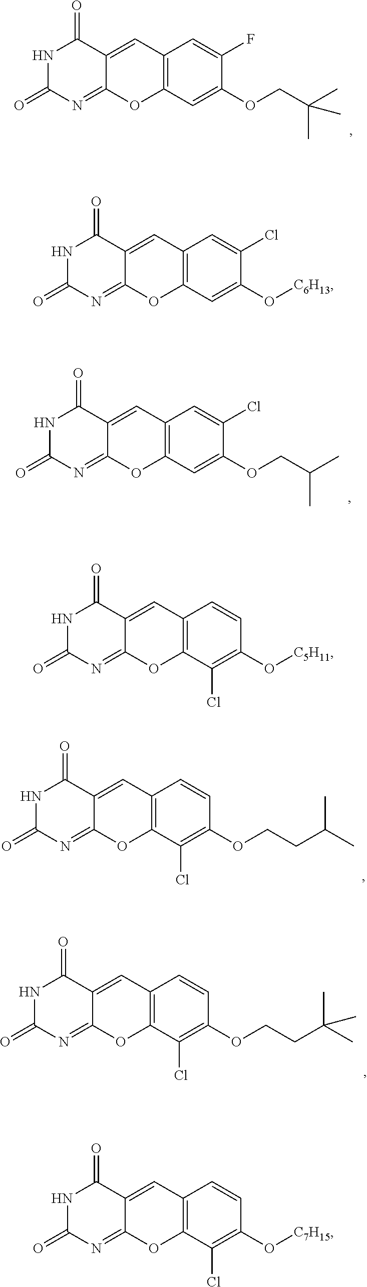 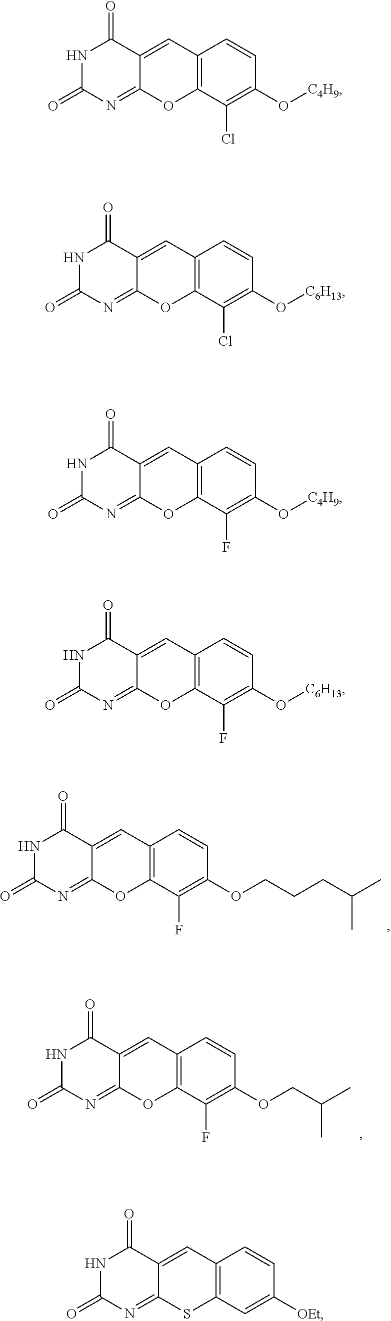 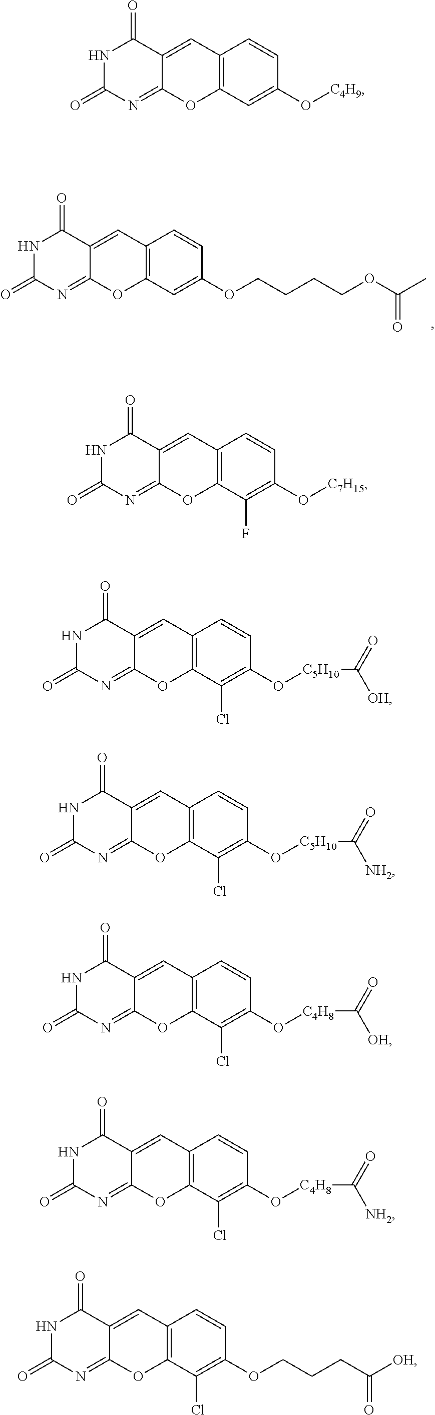 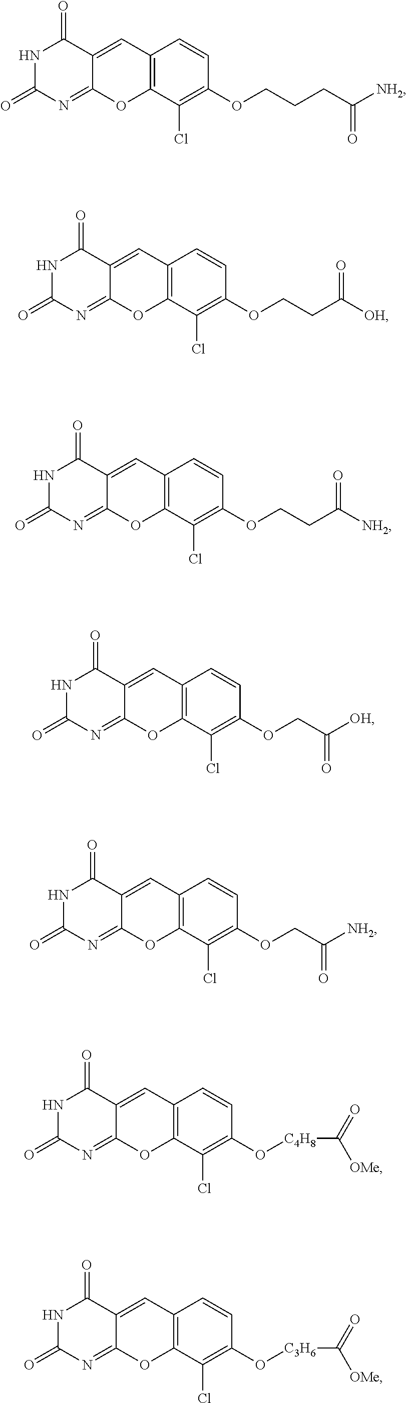 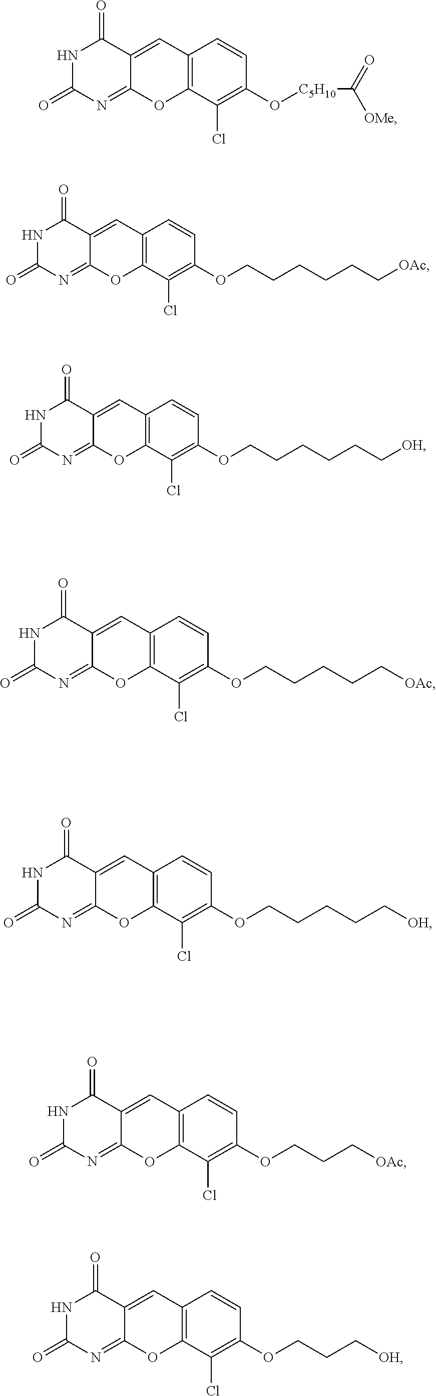 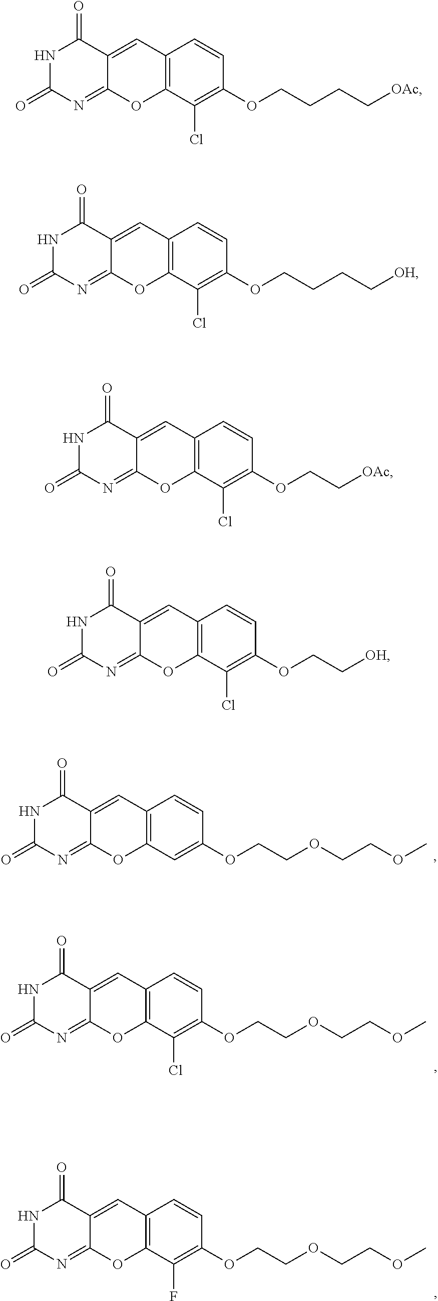 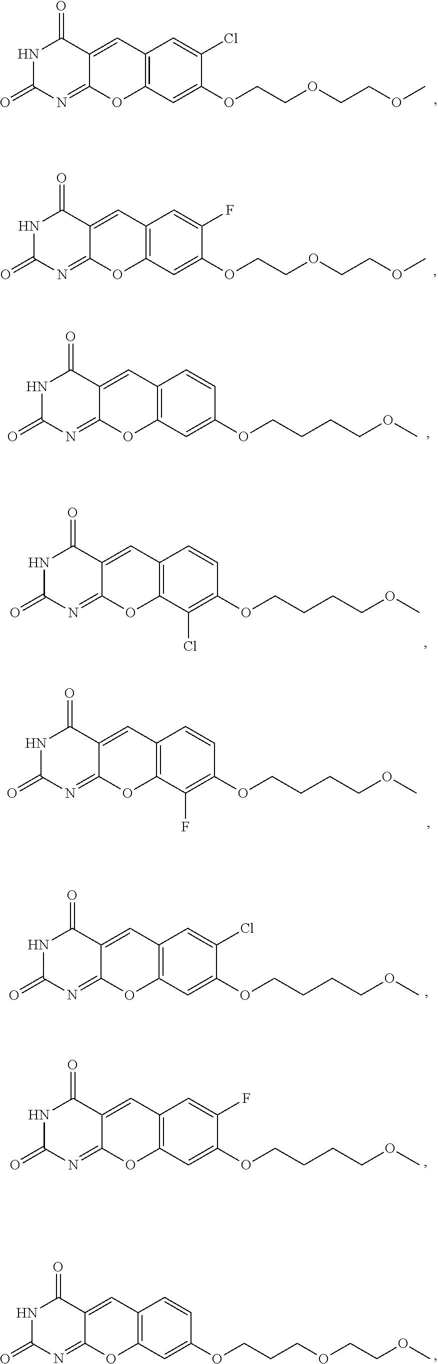 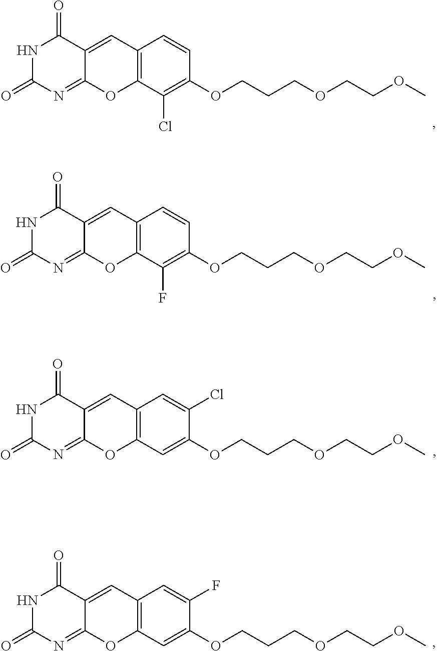 [SMILES: COCCOCCCOc1ccc2cc3c(=O)[nH]c(=O)nc-3oc2c1Cl ; COCCOCCCOc1ccc2cc3c(=O)[nH]c(=O)nc-3oc2c1F ; COCCOCCCOc1cc2oc3nc(=O)[nH]c(=O)c-3cc2cc1Cl ; COCCOCCCOc1cc2oc3nc(=O)[nH]c(=O)c-3cc2cc1F]
and crystalline forms, hydrates, or pharmaceutically acceptable salts thereof.
